(19) United States
(12) Patent Application Publication
(10) Pub. No.: US 2005/0070874 A1
(43) Pub. Date: Mar. 31, 2005

(54) System and method for injecting liquid drug containing biological material

(76) Inventors: Hikaru Matsuda, Ashiya-shi (JP); Yoshiki Sawa, Nishinomiya-shi (JP); Satoshi Taketani, Osaka (JP); Shin Kawamata, Kobe-shi (JP); Shigeru Miyagawa, Osaka (JP); Emi Maeno, Osaka (JP); Yoshiho Toyota, Okazaki-shi (JP); Shinji Ozawa, Nagoya-shi (JP)

Correspondence Address: Brinks Hofer Gilson & Lione;Suite 3600, NBC Tower, P.O. Box 10395, Chicago, IL, 60610, US

(21) Appl. No.: 10/675,254

(22) Filed: Sep. 30, 2003

(51) Int. Cl. A61F 13/00; A61M 31/00

(52) U.S. Cl. 604/500

(57) ABSTRACT

A method for injecting a liquid drug containing a biological material is provided, which comprises the step of injecting the liquid drug containing the biological material contained in an injector into a subject at a predetermined range of velocity.

### BACKGROUND OF THE INVENTION

1. Field of the Invention

The present invention relates to the field of medical technology, particularly regeneration/implantation medicine. More particularly, the present invention relates to a technique of injecting a desired biological material into organisms. The present invention also relates to an apparatus capable of injecting a liquid drug at a constant velocity. More particularly, the present invention relates to a liquid drug injecting apparatus for injecting a liquid containing cells into organisms at a relatively low velocity.

2. Description of the Related Art

With recent advances in medical high-technologies, such as regeneration medicine, implantation medicine, gene medicine, and the like, cells or biological molecules, such as nucleic acid molecules, proteins or the like, are being directly introduced into organisms for therapy, prevention, or the like. Such a technique is considered to become increasingly important in actual clinical situations as regeneration/implantation technologies, gene therapy, and the like are more and more developed.

For example, in the field of the circulatory system, as the number of elderly persons and the number of patients with ischemic heart diseases increases, the number of severe cases for which conventional percutaneous coronary circulation reconstruction cannot be applied also increases. Such severe patients have to be repeatedly hospitalized and released, resulting in high medical cost and deteriorated quality of life. Therefore, there is a demand for development of an epoch-making therapeutic method for the above-described cases.

Treatment results for circulatory disorders are being improved with innovation in internal and surgical therapeutics. However, in the case of highly severe cases, there is no established treatment other than less universal replacement therapy, such as implantation, the use of an artificial organ, and the like. In the current circumstances, it is nearly impossible to prevent the irreversible development of the symptom of organ failure and save lives. In addition, the treatment of such severe cases require high medical cost, such as an expensive fee for treatment itself, a fee for care, and the like, and huge expenses for building infrastructures for patients with sequelae, putting pressure on medical finance in Japan, which has already been deteriorated. Therefore, the establishment and industrialization of any novel, effective and inexpensive (i.e., high cost-performance) treatment, such as regeneration medicine, and the development of basic medical industries supporting it are urgent objectives for medical care as well as for society and the economy. In the world, post-genome biotechnologies are coming into practical use. Also in the circulatory organ field, regeneration medicine has already been realized and the international competition to research and develop it is becoming increasingly fierce. Particularly, the heart, which has been believed to have no regenerating ability, has great value in the application of regeneration medicine. At present, numerous researchers have entered this field and studied vigorously. So-called “regeneration medicine”, which utilizes cells, nucleic acid molecules, or the like to basically repair and regenerate tissue or organs suffering from injury or an intractable disease, which cannot be cured by conventional drug therapy and organ transplantation, is revolutionizing the conventional concept of medical treatment. The establishment of regeneration medicine is now an essential challenge in treating intractable diseases. In the world, regeneration medicine has already been used. In actual medical practice. Further, regeneration medicine is in the initial stage of industrialization mainly in the USA, where the economic merit thereof is recognized and the competition thereof is becoming fierce. In Japan, regeneration medicine is still in the phase of basic research and development mainly in the tissue/cell engineering field. The studies are most recently showing the feasibility of regeneration medicine as medical treatment, but it is pointed out that the studies are not sufficiently associated with industries. In these situations, it is considered to be significantly important to undergo large-scale and comprehensive technological development of regeneration medicine so as to establish the base of a novel high value-added industry in order to activate medical industries in Japan in terms of social importance and urgency.

However, there are various outstanding problems to be solved so as to apply regeneration medicine clinically to humans, including development of cell sources as well as large-scale and safe culture systems, therapeutic devices for cells, and the like. As a cell source for clinical applications, a self-pulsatile cell, a non-self-pulsatile cell, or a muscle cell transformed by gene transfer has been used in animal experiments, in which the call is cultured and the cultured cell is directly injected into a normal or injured cardiac muscle tissue. There has also been a experimental report, that implanted cardiac muscle cells successfully survived in cardiac muscle tissue. In a certain clinical case in which skeletal myoblasts were used in implantation, an improvement in cardiac function was confirmed. At present, cells available for cardiac muscle regeneration therapy include myoblasts derived from self skeletal muscle for use in regeneration of self cardiac muscle, and self bone marrow mononuclear cells. In the case of the latter, clinical studies have already been developed for the inferior limbs and the heart in Japan. However, since the bone marrow mononuclear cell differentiates in various ways, the direct implantation of it has a number of disadvantages, such as poor long-term results and side effects. In view of these points, it is necessary to culture in vitro clinically meaningful graft cells, such as cells differentiated from skeletal myoblasts, mesenchymal stem cells, and the like. As to therapeutic devices, delivery systems have been conventionally studied for, for example, gene therapy and the like. However, most of the research on gene therapy and the like are concentrated into vectors for introducing genes into organisms. Even if vectors having a high affinity to tissue and cells and a high level of introduction efficiency are developed, delivery systems for sending the vectors to affected areas are still required.

It is difficult to implant cells or introduce genes without damaging the cells or the genes. Actually, current gene therapy or cell therapy is mostly carried out by direct injection from the body surface to an affected area using a syringe needle. However, manual direct injection into affected areas using a syringe needle is not considered to have a sufficient therapeutic effect in terms of medical effects. In the above-described therapy, it is believed that a gene within a cell is damaged and the cell nucleus is disrupted, eliciting apoptosis in which the cell dies in accordance with a program.

Conventionally, delivery systems have also been conventionally studied for gene therapy and the like. However, most of the research on gene therapy and the like are concentrated into vectors for introducing genes into organisms. Even if vectors having a high affinity to tissue and cells and a high level of introduction efficiency are developed, delivery systems for sending the vectors to affected areas are still required. Actually, current gene therapy and cell therapy are mostly carried out by direct injection from the body surface to an affected area using a syringe needle.

Various liquid drug injecting devices, such as representatively a syringe and the like, have been conventionally proposed. However, in conventional devices, the injection velocity and acceleration of a liquid drug are not taken into consideration. When a liquid drug is injected manually, the velocity, acceleration, and the like of the liquid drug is relatively high, or vary.

Therefore, conventional devices are not suitable for a device for injecting a liquid drug containing a cell whose activity in the body depends on the velocity, acceleration and the like of the liquid drug during injection. Therefore, there has been a demand for a simple device capable of injecting a liquid drug while maintaining a predetermined velocity unchanged.

### SUMMARY OF THE INVENTION

The above-described problems have been solved by the unexpected achievement that when a biological material is injected into organisms, the biological material is accelerated at a predetermined range of acceleration and thereafter a predetermined range of velocity is maintained.

The present invention was achieved in view of the above-described finding. An object of the present invention is to provide a liquid drug injecting device for injecting a liquid drug while maintaining a predetermined velocity or acceleration thereof unchanged.

Another object of the present invention is to provide a liquid drug injecting device suitable for injection of a cell-containing liquid into the body.

According to one aspect of the present invention, a method is provided for injecting a liquid drug containing a biological material. The method comprises the step of: A) injecting the liquid drug containing the biological material contained in an injector into a subject at a predetermined range of velocity.

In one embodiment of this invention, the predetermined range of velocity maintains a biological activity of the biological material.

In one embodiment of this invention, the predetermined range of velocity is less than or equal to about 20 ml/min.

In one embodiment of this invention, the predetermined range of velocity is less than about 10 ml/min.

In one embodiment of this invention, the predetermined range of velocity is greater than or equal to about 1 ml/min and less than about 10 ml/min.

In one embodiment of this invention, the method further comprises the step of: B) accelerating the liquid drug containing the biological material at a predetermined range of acceleration to reach the predetermined range of velocity.

In one embodiment of this invention, the predetermined range of acceleration maintains a biological activity of the biological material.

In one embodiment of this invention, the predetermined range of acceleration is in the range of about 1 mm/sec² to about 15 mm/sec².

In one embodiment of this invention, an inner diameter of a body of the injector is about 1 mm to about 30 mm, preferably about 3 mm to about 13 mm.

In one embodiment of this invention, an inner diameter of a tip tube of the injector is about 0.1 mm to about 10 mm, preferably about 0.25 mm to about 1 mm.

In one embodiment of this invention, the biological material comprises a material selected from the group consisting of nucleic acid molecules, polypeptides, lipids, sugar chains, small organic molecules and complexes thereof, cells, tissues, and organs.

In one embodiment of this invention, the biological material is a cell, and the velocity is about 1 ml/min to about 20 ml/min.

In one embodiment of this invention, the biological material is a cell, and the velocity is about 1 ml/min to about 10 ml/min.

In one embodiment of this invention, the method further comprises the step of: C) decreasing a velocity of the liquid drug containing the biological material at a predetermined range of acceleration to substantially zero.

In one embodiment of this invention, the absolute value of an acceleration of the decreasing velocity is in the range of about 1 mm to about 15 mm/sec².

In one embodiment of this invention, the injection is carried out for treatment or prophylaxis of a heart.

According to another aspect of the present invention, a method is provided for treating an organ using a liquid drug containing a biological material. The method comprises the step of: A) injecting the liquid drug containing the biological material contained in an injector into a subject at a predetermined range of velocity.

According to another aspect of the present invention, a system is provided for injecting a liquid drug containing a biological material. The system comprises: A) an injector for injecting the liquid drug containing the biological material to a target organism; and B) an adjustor for adjusting the injection of the liquid drug containing the biological material so that the injection velocity of the liquid drug containing the biological material can be maintained within a predetermined range.

In one embodiment of this invention, the predetermined range of velocity maintains a biological activity of the biological material.

In one embodiment of this invention, the predetermined range of velocity is less than or equal to about 20 ml/min.

In one embodiment of this invention, the predetermined range of velocity is less than about 10 ml/min.

In one embodiment of this invention, the predetermined range of velocity is greater than or equal to about 1 ml/min and less than about 10 ml/min.

In one embodiment of this invention, the adjustor can accelerate the liquid drug containing the biological material at a predetermined range of acceleration.

In one embodiment of this invention, the predetermined range of acceleration maintains a biological activity of the biological material.

In one embodiment of this invention, the predetermined range of acceleration is in the range of about 1 mm/sec² to about 15 mm/sec².

In one embodiment of this invention, an inner diameter of a body of the injector is about 1 mm to about 30 mm, preferably about 3 mm to about 13 mm.

In one embodiment of this invention, an inner diameter of a tip tube of the injector is about 0.1 mm to about 10 mm, preferably about 0.25 mm to about 1 mm.

In one embodiment of this invention, the adjustor does not have an adverse influence on a material selected from the group consisting of nucleic acid molecules, polypeptides, lipids, sugar chains, small organic molecules and complexes thereof, cells, tissues, and organs.

In one embodiment of this invention, the biological material is a cell, and the velocity is about 1 ml/min to about 20 ml/min.

In one embodiment of this invention, the biological material is a cell, and the velocity is about 1 ml/min to about 10 ml/min.

In one embodiment of this invention, a cross-sectional area of the injector is about 0.01 mm² to about 1000 mm², preferably about 1 mm² to about 500 mm², and more preferably about 5 mm² to about 150 mm².

In one embodiment of this invention, the injection is carried but for treatment or prophylaxis of a heart.

According to another aspect of the present invention, a system is provided for treating an organ using a liquid drug containing, a biological material. The system comprises: A) an injector for injecting the liquid drug containing the biological material to a target organism; and B) an adjustor for adjusting the injection of the liquid drug containing the biological material so that the injection v locity of the liquid drug containing the biological material can be maintained within a predetermined range.

According to another aspect of the present invention, a liquid drug injecting device is provided, which comprising: a cylinder comprising a nozzle portion at a tip portion thereof, wherein a liquid drug can be loaded into the cylinder and the liquid drug is output through the nozzle portion; and a pushing portion for pushing out the liquid drug contained in the cylinder through the nozzle portion by external control while maintaining a predetermined velocity substantially unchanged.

In one embodiment of this invention, the pushing portion comprises: a plunger provided with a screw-thread portion arranged around an outer perimeter thereof so that the plunger can be moved into the cylinder; and a nut-thread portion provided on an inner wall of the cylinder so that the screw-thread portion of the plunger is engaged with the nut-thread portion.

In one embodiment of this invention, the pushing portion comprises: a plunger arranged so that the plunger can be moved into the cylinder; and a plug provided at a tip portion of the plunger. The plunger comprises a spring-like elastic member which can be compressed when a velocity or acceleration thereof is greater than or equal to a predetermined value.

In one embodiment of this invention, the pushing portion comprises: a plunger provided in the cylinder; and an elastic member provided at a tip portion of the plunger. The elastic member can be compressed when a velocity or acceleration thereof is greater than or equal to a predetermined value.

In one embodiment of this invention, the pushing portion comprises: a plunger provided with a screw-thread portion on an outer perimeter thereof so that the plunger can be moved into the cylinder; a nut-thread portion provided on an inner wall of the cylinder so that the screw-thread portion of the plunger is engaged with the nut-thread portion; and an elastic member provided at a tip portion of the plunger. The liquid drug contained in the cylinder is pushed out with the tip portion of the plunger by rotating the plunger. When the velocity or acceleration of the plunger is greater than or equal to a predetermined value, the elastic member can be compressed.

In one embodiment of this invention, the pushing portion comprises: an inflating member provided on an inner perimeter portion of the cylinder; and a loading portion for loading an incompressible fluid into the inflating member. The incompressible fluid is loaded by the loading portion into the inflating member at a substantially constant velocity and/or acceleration thereof.

In one embodiment of this invention, the pushing portion comprises: a hollow inflating member attached to a rear end portion of the cylinder. The incompressible fluid is loaded by the loading portion into the inflating member at a substantially constant velocity and/or acceleration thereof.

In one embodiment of this invention, the pushing portion comprises: a plunger movably attached to the cylinder; and a driving portion for inserting the plunger into the cylinder at a constant velocity.

In one embodiment of this invention, the liquid drug is a liquid containing a cell.

Functions of the present invention will be described below.

A liquid drug injecting device of the present invention comprises a cylinder into which a liquid drug can be loaded and which comprises a nozzle portion for outputting a liquid drug at a tip portion thereof, and a pushing portion for pushing out the liquid drug contained in the cylinder through the nozzle portion by external control while maintaining a predetermined velocity substantially unchanged. Thereby, it is possible to prevent a predetermined force or more from being exerted on the liquid drug contained in the cylinder during injection of the liquid drug.

Thus, the invention described herein makes possible the advantages of providing (1) a method and system for injecting a liquid drug containing a biological material, such as a gene, a cell, or the like, into organisms efficiently and effectively while maintaining a velocity and/or acceleration thereof unchanged, without damaging the biological material, thereby making it possible to expect a significant improvement in the therapeutic effect of cell implantation therapy, (2) a device for injecting a liquid drug into the body while maintaining a predetermined velocity and/or acceleration unchanged, whereby, for example, when a cell-containing liquid is injected into the body, it is possible to suppress the adverse influence of injection pressure on a cell, and (3) a liquid drug injecting device having a simple structure suitable for injection of a cell-containing liquid into the body.

These and other advantages of the present invention will become apparent to those skilled in the art upon reading and understanding the following detailed description with reference to the accompanying figures.

### BRIEF DESCRIPTION OF THE DRAWINGS

FIG. 1 is an exemplary device according to the present invention which is used in Example 1.

FIG. 2 is a cross-sectional view of a liquid drug injecting device according to an embodiment of the present invention.

FIG. 3 is a schematic cross-sectional view of the liquid drug injecting device of FIG. 2.

FIG. 4 is a cross-sectional view of a liquid drug injecting device according to another embodiment of the present invention.

FIGS. 5A-B are diagrams for explaining a function of the liquid drug injecting device of FIG. 4.

FIG. 6 is a cross-sectional view of a liquid drug injecting device according to still another embodiment of the present invention.

FIG. 7 is a diagram for explaining a function of the liquid drug injecting device of FIG. 6.

FIG. 8 is a cross-sectional view of a liquid drug injecting device according to still another embodiment of the present invention.

FIG. 9 is a cross-sectional view of a liquid drug injecting device according to still another embodiment of the present invention.

FIG. 10 is a diagram for explaining a function of the liquid drug injecting device of FIG. 9.

FIG. 11 is a cross-sectional view of a liquid drug injecting device according to still another embodiment of the present invention.

FIG. 12 is a diagram for explaining a function of the liquid drug injecting device of FIG. 11.

FIG. 13 is a cross-sectional view of a liquid drug injecting device according to still another embodiment of the present invention.

FIG. 14 is a cross-sectional view of a liquid drug injecting device according to still another embodiment of the present invention.

FIG. 15 is an exemplary structure of a liquid drug injecting system according to the present invention.

FIG. 16 is a graph showing a detailed example of the injection time and linear velocity of a system according to the present invention.

FIG. 17 is a graph showing a detailed example of the injection time and linear velocity when a 1-ml syringe was manually used at a rate of 38 ml/min.

FIGS. 18A-D are graphs showing typical exemplary cell proliferation rates over time after injection using the MTT method.

FIG. 19 is a graph showing other exemplary cell proliferation rates over time after injection using the MTT method.

FIG. 20 is a graph showing an exemplary result of Example 4.

FIG. 21 is a diagram showing devices used in Example 5.

FIG. 22 is a diagram showing that a device is inserted into the heart.

FIG. 23 is a diagram showing that a device is inserted into the heart.

FIG. 24 is a diagram showing the results of an experiment in Example 5.

### DESCRIPTION OF THE PREFERRED EMBODIMENTS

It should be understood throughout the present specification that articles for singular forms include the concept of their plurality unless otherwise mentioned. Therefore, articles or adjectives for singular forms (e.g., “a”, “an”, “the”, and the like in English) include the concept of their plurality unless otherwise specified. Also, it should be also understood that terms as used herein have definitions ordinarily used in the art unless otherwise mentioned. Therefore, all technical and scientific terms used herein have the same meanings as commonly understood by those skilled in the relevant art. Otherwise, the present application (including definitions) takes precedence.

(Definitions)

As used herein, the terms “biological molecule” and “biological material” are used interchangeably and each refers to a material related to organisms. Particularly, a sample containing such a biological material may herein refer to a biological sample. As used herein, the term “organism” refers to a biological system, including, but not limited to, animals, plants, fungi, viruses, and the like. Therefore, the biological molecule and the biological materials include, but are not limited to, materials extracted from organisms. Any molecule capable of affecting an organism falls within the definition of the biological molecule and the biological material. The biological molecule and the biological material include cells, tissues, a part or the whole of organs, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. As used herein, the biological molecule may be preferably a cell, a polypeptide or a polynucleotide which have a medical effect.

(Cells)

The term “cell” herein is used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be naturally-occurring cells or artificially modified cells (e.g., fusion cells, genetically modified cells, etc.). A gene for use in genetic modification may have efficacy as it is or as it is expressed. Example of a source for cells include, but are not limited to, a single cell culture, the embryo, blood, or body tissue of a normally grown transgenic animal, a cell mixture, such as cells from a normally grown cell line, and the like.

Cells used herein may be derived from any organism (e.g., any unicellular organism (e.g., bacteria, yeast, etc.) or any multicellular organism, (e.g., animals (e.g., vertebrates and invertebrates), plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialla, edentate, dermoptera, chiroptera, carnivore, insectivore, probosoidea, perissodactyla, artiodactyla, tubulidentata, pholidota, sirenia, cetacean, primates, rodentia, lagomorpha, etc.). In one embodiment, cells derived from Primates (e.g., chimpanzee, Japanese monkey, human) are used. Particularly, cells derived from a human are used. The present invention is not limited to this. Cells used herein may be stem cells or somatic cells. Such cells may be used for the purpose of implantation. Preferably, cells are suitable for the organisms which are intended to be subjected to injection. More preferably, cells are isologous to the organism. Even more preferably, cells are derived from the organism in view of immune reactions.

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cell, tissue-specific stem cell, or somatic stem cell). A stem cell may be an artificially produced cell as long as it can have the above-described abilities. The term “embryonic stem cell” refers to a pluripotent stem cell derived from early embryos. As are different from embryonic stem cells, the direction of differentiation of tissue stem cells is limited. Embryonic stem cells are located at specific positions in tissues and have undifferentiated intracellular structures. Therefore, tissue stem cells have a low level of pluripotency. In tissue stem cells, the nucleus/cytoplasm is high and there are little intracellular organelles. Tissue stem cells generally have pluripotency and the cell cycle is long, and can maintain proliferation ability beyond the life of an individual. Stem cell used herein may be embryonic stem cells or tissue stem cells as long as they are employed for intended treatment.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreas (common) stem cells, liver stem cells, and the like. Tissue stem cells in the bone marrow system include hematopoeitic stem calls, mesenchymal stem cells, and the like. Tissue stem cells in the nervous system include neural stem cells, retina stem cells, and the like.

As used herein, the term “pluripotency” refers to a nature of a cell, i.e., an ability to differentiate into one or more, preferably two or more, tissues or organs. Therefore, the term “pluripotent” is herein used interchangeably with “undifferentiated” unless otherwise mentioned. Typically, the pluripotency of a cell is limited as the cell is developed, and in an adult, cells constituting a tissue or organ rarely alter to different cells, where the pluripotency is last. Particularly, epithelial cells are unlikely to alter to other epithelial cells. However, such alteration typically occurs in pathological conditions, and is called metaplasia. However, mesenchymal cells tend to easily undergo metaplasia, i.e., alter to other mesenchymal cells, with relatively simple stimuli. Therefore, mesenchymal cells have a high level of pluripotency. ES cells have pluripotency. Tissue stem cells have pluripotency. As used herein, the term “totipotency” refers to the pluripotency of a cell, such as a fertilized egg, to differentiate into all cells constituting an organism. Thus, the term “pluripotency” may include the concept of totipotency. An example of an in vitro assay for determining whether or not a cell has pluripotency, includes, but is not limited to, culture under conditions for inducing the formation and differentiation of embryoid bodies. Examples of an in vivo assay for determining the presence or absence of pluripotency, include, but are not limited to, implantation of a cell into an immunodeficient mouse so as to form teratoma, injection of a cell into a blastocyst so as to form a chimeric embryo, implantation of a cell into a tissue of an organism (e.g., injection of a cell into ascites) so as to undergo proliferation, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they can achieve the intended treatment.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoetic stem cells, mesenchymal stem cells, and the like. Stem cells of mesoderm origin are mostly present in organs, including liver stem cells, pancreas stem cells, and the like.

As used herein, the term “established” in relation to cells refers to a state of a cell in which a particular property (pluripotency) of the cell is maintained and the cell undergoes stable proliferation under culture conditions. Therefore, established stem cells maintain pluripotency. Both established cells and primary culture cells may be herein used.

As used herein, the term “differentiated cell” refers to a cell having a specialized function and form (e.g., muscle cells, neurons, etc.). Unlike stem cells, differentiated cells have no or little pluripotency. Examples of differentiated cells include epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like. Cells used herein may be any of the above-described cells as long as they can be used to carry out the intended treatment. As used herein, the terms “differentiation” or “cell differentiations” refers to a phenomenon that two or more types of cells having qualitative differences in form and/or function occur in a daughter cell population derived from the division of a single cell. Therefore, “differentiation” includes a process during which a population (family tree) of cells which do not originally have a specific detectable feature acquire a feature, such as production of a specific protein, or the like.

As used herein, the term “tissue” refers to an aggregate of cells having substantially the same function and/or form in a multicellular organism. “Tissue” is typically an aggregate of cells of the same origin, but may be an aggregate of cells of different origins as long as the cells have the same function and/or form. Therefore, when cells are injected into a tissue according to the present invention, the tissue may be composed of an aggregate of cells of two or more different origins. Typically, a tissue constitutes a part of an organ. Animal tissues are separated into epithelial tissue, connective tissue, muscular tissue, nervous tissue, and the like, on a morphological, functional, or developmental basis. Plant tissues are roughly separated into meristematic tissue and permanent tissue according to the developmental stage of the cells constituting the tissue. Alternatively, tissues may be separated into single tissues and composite tissues according to the type of cells constituting the tissue. Thus, tissues are separated into various categories. Any tissue may be herein subjected to injection as long as the tissue can be subjected to the intended treatment.

Any organ or a part thereof may be used as a biological material to be injected in the present invention. Tissues or cells to be injected in the present invention may be derived from any organ. As used herein, the term “organs” refers to a morphologically independent structure localized at a particular portion of an individual organism in which a certain function is performed. In multicellular organisms (e.g., animals plants), an organ consists of several tissues spatially arranged in a particular manner, each tissue being composed of a number of cells. An example of such an organ includes an organ relating to the vascular system. In one embodiment, organs targeted by the present invention include, but are not limited to, skin, blood vessel, cornea, kidney, heart, liver, umbilical cord, intestine, nerve, lung, placenta, pancreas, brain, peripheral limbs, retina, and the like.

A cell to be injected in the present invention can be cultured in a medium suitable for the cell and with a culture method. The medium and culture method can be prepared with the following techniques well-known in the art. An illustrative medium includes, but is not limited to, Dulbecco's Modified Eagle's Medium (DMEM) (see, H. Eagle, Science 122:-501 (1955); R. Dulbecco, G. Freeman, Virology, 8, 396, 1959). As an illustrative culture method, any technique can be used. For example, see, T. Kono, Biophys. Acta, 178, 397(1969) for separation of adult ventricular muscle with collagenase treatment; K. Goshima, J. Mol. Cell Cardiol., 8., 217(1976) for separation and culture of juvenile cardiac muscle cells; I. E., Konigsberg, Science, 140, 1273 (1963) for culture of skeletal muscular cells; Yasusada Miura, Ketsueki Kan Saibo [blood stem cell], Chugai-Igaku-Sha, Tokyo, 1983 for culture of bone marrow cells; Pluzenik, D. H. & Saches, J. Cell. Comp. Physiol., 66, 319(1965), and Bredley, T. R., & Metcalf, D., Aust. J. Exp. Biol. Med. Sci., 44, 287 (1966) for culture by colony formation; and Dexter, T. M., Allen, T. D., & Lajtha, L. G., J. Cell. Physiol., 91, 335(1977) for a long-term proliferation culture method by interaction between hematopoietic stem cells and mesenchymal stem cells.

(Other Biological Materials)

The injection method of the present invention can be applied to other biological materials.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylatlon, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. A polypeptide for use in the present invention can exhibit at least one biological activity in an organism into which the polypeptide; is injected, preferably a pharmaceutical effect.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise, indicated, a particular nucleic acid sequence also implicitly encompasses conservatively-modified variants thereof (e.g. degenerate codon substitutions) and complementary sequences and as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsika et al., J. Biol. Chem. 260:2605-2608 (1985); Rossolini et. al., Mol. Cell. Probes 8:91-98(1994)). As used herein, the term “nucleic acid molecule” is also used interchangeably with the terms “nucleic acid”, “oligonucleotide”, and “polynucleotide”, including cDNA, mRNA, genomic DNA, and the like. A polypeptide for use in the present invention can exhibit at least one biological activity in an organism into which the polypeptide is injected, preferably a pharmaceutical effect. Alternatively, a polynucleotide for use in the present invention may exhibit at least one biological activity in an organism, into which the polypeptide is injected, if it is transcribed and/or translated, preferably a pharmaceutical effect.

As used herein, nucleic acid and nucleic acid molecule may be included by the concept of the term “gene”. A nucleic acid molecule encoding the sequence of a given gene includes “splice mutant (variant).” Similarly, a particular protein encoded by a nucleic acid encompasses any protein encoded by a spice variant of that nucleic acid. “Splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, the gene of the present invention may include the splice mutants herein. As used herein, “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule”, and/or “protein”, “polypeptide”, “oligopeptide” and “peptide”, which are expressed by a gene. Those skilled in the art understand what a gene product is, according to the context. The similarity, identity and homology of amino acid sequences and base sequences are herein compared using FASTA (sequence analyzing tool) with the default parameters.

Amino acids may be referred to herein by either their commonly known three letter symbols or by the one-letter symbols recommended by the, IUPAC-IUB Biochemical Nomenclature Commission. Nucleotides, likewise, may be referred to by their commonly accepted single-letter codes.

As used herein, the term “corresponding” gene refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, a gene corresponding to a mouse kinase gene or the like can be found in other animals (human, rat, pig, cattle, and the like). Such a corresponding gene can be identified by a technique well known in the art. Therefore, for example, a corresponding gene in a given animal can be found by searching a sequence database of the animal (e.g., human, rat) using the sequence of a reference gene (e.g., a mouse kinase gene, or the like) as a query sequence.

As used herein, the terms “fragment” and “part” are used interchangeably and each term, when referring to the entirety of a certain biological material (a full-length polypeptide, a full-length polynucleotide, a whole organ, a whole cell, etc.), refers to a part thereof. Therefore, the term “fragment” and “apart” with respect to a polypeptide or polynucleotide refer to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification. The length of a useful fragment may be determined depending on whether or not at least one function is maintained among the functions of a full-length protein which is a reference of the fragment. A biological material to be injected in the present invention, such as a polypeptide, a polynucleotide, or the like, may be a fragment or a part thereof.

As used herein, the term “compound” refers to any identifiable chemical substance or molecule, including, but not limited to, a low molecular weight molecule, a peptide, a protein, a sugar, a nucleotide, or a nucleic acid. Such a compound may be a naturally-occurring product or a synthetic product. A compound to be injected in the present invention may be any compound, preferably a compound which exhibits a biological activity and/or pharmaceutical effect when it is injected into organisms.

As used herein, the term “complex molecule” refers to a molecule in which a plurality of molecules, such as polypeptides, polynucleotides, lipids, sugars, small molecules, or the like, are linked together. Examples of a complex molecule include, but are not limited to, glycolipide, glycopeptides, and the like. A biological material to be injected in the present invention may be may be a complex molecule.

As used herein, the term “isolated” in relation to a biological element (e.g., nucleic acid, protein, or the like) means that the biological element is substantially separated or purified from other biological elements in cells of a naturally-occurring organism (e.g., in the case of nucleic acids, elements other than nucleic acids or nucleic acids having nucleic acid sequences other than an intended nucleic acid; and in the case of proteins, elements other than proteins or proteins having amino acid sequences other than an intended protein). The “isolated” nucleic acid and protein include nucleic acids and proteins purified by a standard purification method. The isolated nucleic acids and proteins also include chemically synthesized nucleic acids and proteins. A biological material to be injected in the present invention may be isolated in this manner.

As used herein, the term “purified” in relation to a biological element (e.g., nucleic acids, proteins, and the like) means that at least a part of the naturally accompanying elements is removed from the biological element. Therefore, ordinarily, the purity of the biological element of a purified biological element is higher than the biological element in a normal state (i.e., concentrated). A biological material to be injected in the present invention may be purified in this manner.

As used herein, the terms “purified” and “isolated” mean that the same type of biological element is present at preferably at least 75% by weight, at more preferably at least 85% by weight, at even more preferably at least 95% by weight, and at most preferably at least 98% by weight.

As used herein, the term “biological activity” refers to activity possessed by an element (e.g., a polynucleotide, a protein, etc.) within an organism, including activities exhibiting various functions (e.g., transcription promoting activity, proliferation activity, cell division activity, etc.). For example, when two elements interact with each other, the biological activity includes binding of the two molecules and a biological change due to the binding. For example, when one molecule is precipitated using antibodies, another molecule may also precipitate. In this case, It is determined that the two molecules are bound together. Therefore, observation of such coprecipitation provides a determination method, for example. Specifically, for example, when a certain factor is an enzyme, the biological activity thereof includes its enzyme activity. In another example, when a certain factor is a ligand, the biological activity thereof includes the binding to a receptor corresponding to the ligand. The above-described biological activity can be measured by techniques well-known in the art.

As used herein, the term “maintain biological activity” in relation to a biological material, means that at least one type of biological activity as defined above of the biological material is maintained at at least about 50% compared to the biological activity when the biological material was prepared. The value of a desired biological activity is based on values determined by a commonly used assay. Therefore, in the case of a polynucleotide to be expressed, the expression frequency of the polynucleotide is used as an index, or in the case of a cell to be injected, the proliferation activity of the cell is used as an index. In the case of a polypeptide, the activity (e.g., enzyme activity) of the polypeptide is used as an index. The above-described biological activity can be measured by techniques well-known in the art. Based on the measured values, the maintenance of the biological activity may be determined.

(General Techniques in Biochemistry, Molecular Biology, and Cell Biology)

Molecular biological techniques, biochemical techniques, microorganism techniques, and cellular biological techniques as used herein are well known in the art and commonly used, and are described in, for example, Sambrook J. et al. (1989), Molecular Cloning: A Laboratory Manual, Cold Spring Harbor and its 3rd Ed. (2001): Ausubel, F. M. (1987), Current Protocols in Molecular Biology, Greene Pub. Associates and Wiley-Interscience; Ausubel, F. M. (1989) Short Protocols in Molecular Biology: A Compendium of Methods from Current Protocols in Molecular Biology, Greene Pub. Associates and Wiley-Interscience: Innis, M. A. (1990), PCR Protocols: A Guide to Methods and Applications, Academic Press: Ausubel, F. M. (1992), Short Protocols In Molecular Biology: A Compendium of Methods from Current Protocols in Molecular Biology, Greene Pub. Associates; Ausubel, P. M. (1995), Short Protocols in Molecular Biology: A Compendium of Methods from Current Protocols in Molecular Biology, Greene Pub. Associates; Innis, M. A. et al. (1995,) PCR. Strategies, Academic Press; Ausubel, F. M. (1999), Short Protocols in Molecular Biology: A Compendium of Methods from Current Protocols in Molecular Biology, Wiley, and annual updates: Sninsky, J. J. et al., (1999), PCR Applications: Protocols for Functional Genomics, Academic Press; Hogan, B. et al. , Manipulating the mouse embryo, a laboratory manual, 2nd ed., Cold Spring Harbor Press, New York, 1994; Gosden, R. G., Fetal Transplants in Medicine (Edward, R. G. ed.), Cambridge Univ. Press, 1992: Special Issue,. Jikken Igaku [Experimental Medicine] “Idenshi Donyu & Hatsugen Kaiseki Jikkenho [Experimental Methods for Gene Introduction & Expression Analysis]”, Yodo-sha, 1997; Noro Nakatsuji ed., Kansaibo Kuron Kenkyu Purotokoru [Protocols for Stem cell and Clone Research, Yodo-sha (2001), and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985,), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approac, IRL Press: Adams, R. L. et al. (1992), The Biochemistry of the Nucleic Acids, Chapman & Hall, Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

(Therapeutic/Prophylactic Administration and Composition)

The present invention provides a method for effectively administering a biological material to a subject. The biological material can be mixed to a composition. The composition may be a therapeutic composition, a treatment composition, a prophylactic composition, or the like. In a preferred aspect, a compound may be substantially purified (e.g., a compound is substantially free from a material which limits the effect of the compound or has an undesirable side effect).

As used herein, the terms “diagnostically effective amount”, “prophylactically effective amount”, “treatment (or therapeutically) effective amount”, and “prognostically effective amount” refer to an amount medically effective in diagnosis, prophylaxis, treatment (or therapy), and prognosis, respectively. These amounts can be determined by those skilled in the art using techniques well known in the art in view of various parameters (e.g., a subject's condition, a disease condition, a biological material to be administered, a medium for a biological material (e.g., a culture medium, a buffer, etc.), an injecting device, etc.).

As used herein, the term “improve” in relation to a cardiac function means that when a cell is administered into an organism (e.g., a coronary artery) with a method of the present invention, the following phenomenon occurs by 5 weeks after implantation (preferably, by 2 weeks after implantation), for example: an increase in the density of blood capillary in an implanted region by a factor of at least 1.5, preferably at least 2; a decrease in a transmural cicatrix region by at least 5%, more preferably at least 10%, 15%, 20%, or 25%; an increase in the thickness of a transmural cicatrix by at least 5%, preferably at least 10%, 15%, 20%, or 25%; a decrease in left ventricle volume/weight ratio by at least 5%, more preferably at least 10%, 15%, 20%, 25%, or 30%; an increase in the systolic pressure or the diastolic pressure by at least 5%, more preferably at least 10% or 15%; or an increase in ejection fraction by at least 5%, preferably at least 10%, 15%, 20%, or 25%.

In addition to heart, the effect of the present invention can be assessed for other targets to be treated by the present invention using criteria well known in the art depending on the target. Examples of targets other than heart include, but are not limited to, cranial nerve, lung, liver, foot, leg, and bedsores. Examples of a publication to be referenced so as to select an assessment criterion include, but are not limited to, standard lieterature, such as Merck Manual (up-to-date version) and the like. Specifically, the assessment is performed as follows.

Implantation of bone marrow cells may be applied to the following patients having peripheral vascular diseases (chronic arteriosclerosis obliterans, Buerger's disease): patients in stages III and IV of the Fontaine classification, and grade II, category 4 and grade III, categories 5 and 6 of the Rutherford's clinical classification of chronic ischemic extremity, who suffer from pain at rest or ischemic ulcer/necrosis, have severe impairment of quality of life so that circulation reconstruction cannot be applied thereto, and amputation is expected in the future.

For example, Matsubara et al. has inaugurated a project team for “J-TACT: Japan trial of Therapeutic Angiogenesis by Cell Transplantation of marrow-derived cells for patient with ischemic heart disease (angina pectoris, myocardial infarction) and peripheral artery disease (ASO, Buerger's disease)” which is collaborative research in which five universities have participated. The team has carried out the method for 11 ASO patients (6 complicated with diabetes: 3 with dialysis) since July, 2002. All of the patients had ischemic lower extremity of Fontaine III or IV, which had not been improved by surgical or internal therapy. The patients had no inadequately controlled diabetes, retinopathy, or malignant tumor.

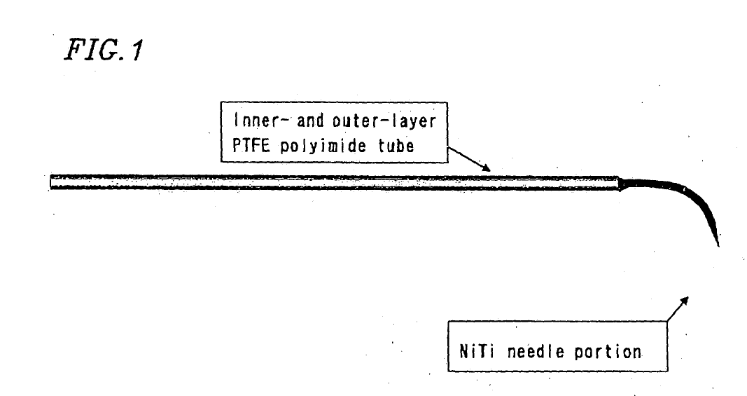

500-700 ml of automarrow liquid was collected under general anesthesia. Thereafter, bone marrow mononuclear cells were isolated. About a billion calls were injected in portions into about 40 sites of the muscle of the ischemic lower extremity. As a result, the blood pressure ratio of the upper arm and the lower extremity (ABPI: ankle brachial pressure index) was significantly increased from mean 0.26 before transplantation to 0.41 after 28 days from transplantation (FIG. 1). Among the 11 patients, 8 patients had an increase in ABPI by 0.1 or more (the clinical category “improved” defined by Society for Vascular Surgery. Pain at rest in the ischemic lower extremity was completely extinguished in 10 of the 11 patients. Painless walking time was increased from 2.0 min to 5.3 min. i.e., by a factor of 2.5 or more. Plethysmography showed a significant increase in blood flow in the lower extremity from 16.1 mL/min to 36.5 mL/min, i.e., by a factor of 2 or more. Skin temperature was also significantly increased from 28.5° C. to 30.0° C.

Inflammation and rubor were not observed at the transplantion locations. Blood VEGF, HGF, and FGF concentrations, the number of leukocytes, and the number of platelets were not changed.

In the phase II trial, randomized double-blind control was carried out. ABPI was increased by 0.97 in the bone marrow transplanted group, showing a significant difference compared to ABPI 0.024.of the control peripheral blood mononuclear cell group.

According to the above-described results, it is considered that transplantation of automarrow cells into ischemic lower extremity is a safe and effective vascularization therapy.

Fontaine Classification: the severity of arteriosclerosis obliterans is divided into stages I to IV, depending on the symptom.

Stage I: The lowest grade. “Chill and numbness” appear in limbs.

Stage II: A symptom “intermittent claudication” appears, in which if a patient walks a certain distance, pain takes place in the legs so that the patient cannot walk; however, the patient rests for a while and then can resume walking. The muscle of the legs requires a larger amount of oxygen upon walking than at rest. If arteriosclerosis is present in the blood vessel of legs, a sufficient amount of blood cannot be transferred into the muscle which in turn lacks oxygen and causes pain. The continuous walking distance depends on the degree of blood flow impairment. If the degree is relatively low, the walking distance is 200-300 m. If the symptom proceeds, the maximal walking distance is about 50 m.

Stage III. “Pain at rest”. Even when a patient stays at rest, pain occurs in the limbs due to lack of blood flow in peripheral tissues. Pain may prevent sleep.

Stage IV: The highest stage. Ulcer or necrosis takes place in sites having blood flow impairment. Pain is severe. The disease may worsen to such an extent that bones, tendons of muscle, or the like may be exposed.

API: A test for determining the severity of arteriosclerosis obliterans present in the legs based on a ratio of the blood pressure of a foot and the blood pressure of a hand. The blood pressure of a foot is normally 1.0-1.2 times higher than the blood pressure of a hand. If arteriosclerosis is present in a blood vessel of a foot, the blood pressure of the foot is lowered. If the ratio is about 0.8, intermittent claudication (stage II) is likely to occur. If about 0.6, pain at rest (stage III) is likely to occur. If about 0.4, ulcer or necrosis (stage IV) is likely to occur.

Thermography: A test for measuring skin temperature. The presence or absence of blood flow impairment or sites of impairment are revealed by temperature variations.

Animals targeted by the present invention include any organism as long as it can be used for the purpose of administration of a biological material (e.g. animals (e.g., vertebrates, invertebrate)). Preferably, the animal is a vertebrate (e.g., Myxiniformes, Petronyzoniformes, Chondrichthyes, Osteichthyes, amphibian; reptilian, avian, mammalian, etc.), more preferably mammalian (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.). Illustrative examples of a subject include, but are not limited to, animals, such as cattle, pigs, horses, chickens, cats, dogs, and the like. More preferably, Primates (e.g., chimpanzee, Japanese monkey, human, etc.) are used. Most preferably, a human is used.

When a cell, a nucleic acid molecule or polypeptide of the present invention is used as a medicament, the medicament may further comprise a pharmaceutically acceptable carrier (e.g., a culture medium in the case of a cell). Any pharmaceutically acceptabl carrier known in the art may b used in the medicament of the present invention.

Suitable formulation materials or pharmaceutically acceptable agents include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. Representatively, a medicament of the present invention may be administered in the form of a composition additionally comprising an active ingredient (e.g., a cell), at least one physiologically acceptable carrier, an excipient, or a diluent. For example, a suitable vehicle may be water for injection, physiological saline solution, or artificial cerebrospinal fluid, possibly supplemented with other materials common in compositions for parenteral administration.

Acceptable carriers, excipients or stabilizers used herein preferably are nontoxic to recipients and are preferably inert at the dosages and concentrations employed, and preferably include buffers such as phosphate, citrate, or other organic acids, ascorbic acid, α-tocophenol; low molecular weight polypeptides; proteins (e.g., serum albumin, gelatin, or immunoglobulins); hydrophilic polymers (e.g., polyvinylpyrrolidone); amino acids (e.g., glycine, glutamine, aspargine, arginine or lysine); monosaccharides, disaccharides, and other carbohydrates (including glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Neutral buffered saline or saline mixed with serum albumin are exemplary appropriate carriers. Preferably, the product is formulated as a lyophilizate using appropriate excipients (e.g., sucrose). Other standard pharmaceutically acceptable carriers, diluents, and excipients may be included as desired. Other exemplary compositions comprise Tris buffer of about pH 7.0-8.5, or acetate buffer of about pH 4.0-5.5 ,which may further include sorbitol or a suitable substitute therefor.

Hereinafter, commonly used preparation methods of the medicament of the present invention will be described. Note that animal-drug compositions, quasi-drugs, marine drug compositions, food compositions, cosmetic compositions, and the like are prepared using known preparation methods.

The cell, polypeptide, polynucleotide and the like of the present invention can be optionally mixed with a pharmaceutically acceptable carrier and can be parenterally administered as liquid formulations (e.g., injections, suspensions, solutions, spray agents, etc.). Examples of pharmaceutically acceptable carriers include excipients, lubricants, binders, disintegrants, disintegration inhibitors, absorption promoters, adsorbers, moisturizing agents, solvents, solubilizing agents, suspending agents, isotonic agents, buffers, soothing agents and the like. Additives for formulations, such as antiseptics, antioxidants, colorants, sweeteners, and the like can be optionally used. The composition of the present invention can be mixed with substances other than cells, polynucleotides, polypeptides, and the like.

Examples of excipients include glucose, lactose, sucrose, D-mannitol, crystallized cellulose, starch, calcium carbonate, light silicic acid anhydride, sodium chloride, kaolin, urea, and the like.

Examples of absorption, promoters include, but are not limited to, quaternary ammonium salts, sodium lauryl sulfate, and the like.

Examples of stabilizers include, but are not limited to, human serum albumin, lactose, and the like.

Preferable examples of solvents in liquid formulations include injection solutions, alcohols, propyleneglycol, macrogol, sesame oil, corn oil, and the like.

Preferable examples of solubilizing agents in liquid formulations include, but are not limited to, polyethyleneglycol, propyleneglycol, D-mannitol, benzyl benzoate, ethanol, trisaminomethane, cholesterol, triethanolamine, sodium carbonate, sodium citrate, and the like.

Preferable examples of suspending agents in liquid formulations include surfactants (e.g., stearyltriethanolamine, sodium lauryl sulfate, lauryl amino propionic acid, lecithin, benzalkonium chloride, benzethonium chloride, glycerin monostearate, etc.), hydrophilic macromolecule (e.g., polyvinyl alcohol, polyvinylpyrrolidone, carboxymethylcellulose sodium, methylcellulose, hydroxymethylcellulose, hydroxyethylcellulose, hydroxyethylcellulose, hydroxypropylcellulose, etc.), and the like.

Preferable examples of isotonic agents in liquid formulations include, but are not limited to, sodium chloride, glycerin, D-mannitol, and the like.

Preferable examples of buffers in liquid formulations include, but are not limited to, phosphate, acetate, carbonate, citrate, and the like.

Preferable examples of soothing agents in liquid formulations include, but are not limited to, benzyl alcohol, benzalkoniumchloride, procaine hydrochloride, and the like.

Preferable examples of antiseptics in liquid formulations include, but are not limited to, parahydroxybenzoate esters, chlorobutanol, benzyl alcohol, 2-phenylethylalcohol, dehydroacetic acid, sorbic acid, and the like.

When a cell is prepared as a formulation, the formulation may be supplemented with a pharmaceutical agent for supporting the survival of the cell (e.g., a carbohydrate, a cytokine, a vitamin, etc.).

Preferable examples of antioxidants in liquid formulations include, but are not limited to, sulfite, ascorbic acid, α-tocopherol, cysteine, and the like.

When liquid agents and suspensions are prepared as injections, they are sterilized and are preferably isotonic with the blood or a medium at an injection site for other purposes. Typically, these agents are made aseptic by filtration using a bacteria-retaining filter or the like, mixing with a bactericide or, irradiation, or the like. Following this treatment, these agents may be made solid by lyophilization or the like. Immediately before use, sterile water or sterile injection diluent (lidocaine hydrochloride aqueous solution, physiological saline, glucose aqueous solution, ethanol or a mixture solution thereof, etc.) may be added.

The medicament composition of the present invention may further comprise a colorant, a preservative, an aroma chemical, a flavor, a sweetener, or other drugs.

In another embodiment of the present invention, injection may be carried out intravenously or subcutaneously. When systemically administered, a medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art.

Administration methods may be herein any parenteral administration (e.g., intravenous, intramuscular, subcutaneous, intradermal, mucosal, intrarectal, vaginal, topical to an affected site, to the skin, etc.). A prescription for such administration may be provided in any formulation form. Such a formulation form includes liquid formulations, injections, sustained-released preparations, and the like.

In the present invention, examples of parenteral routes of administration include, but are not limited to, intra-coronary arterial injection, intravenous injection intra-arterial injection, intraperitoneal injection, subcutaneous injection, intramuscular injection, intranasal, intra-rectal, intra-vaginal, transdermal, intra-billiary, intra-pancreatic duct, and the like. Preferably, intra-coronary arterial injection may be selected.

The medicament of the present invention may be prepared for storage by mixing a sugar chain composition having the desired degree of purity with optional physiologically acceptable carriers, excipients, or stabilizers (Japanese Pharmacopeia ver. 14, or a supplement thereto or the latest version; Remington's Pharmaceutical Sciences, 18th Edition, A. R. Gennaro, ed., Mack Publishing Company, 1990; and the like), in the form of lyophilized cake or aqueous solutions.

Various known delivery systems may be employed (e.g., liposomes, microparticles, microcapsules, etc.). Biological materials may be administered by any convenient route (e.g., by infusion or bolus injection), and may be administered together with other biologically active agents. Administration can be systemic or local. In addition, biological materials can be introduced into the central nervous system by any suitable route (including intraventricular and, intrathecal injection; intraventricular injection may be facilitated by an intraventricular catheter, for example, attached to a reservoir, such as an Ommaya reservoir). When administration into a coronary artery is desired, an illustrative system of the present invention as shown in FIG. 1 can be employed, in which a NiTi needle is connected to a polyimide tube (preferably, coated with PTFE).

In a specific embodiment, it may be desirable to administer a biological material, such as a cell, a polypeptide, a polynucleotide or the like, or a composition thereof locally to the area in need of treatment (e.g., the heart, a coronary artery, etc.); this may be achieved by, for example, and not by way of limitation, local infusion during surgery, topical application (e.g., in conjunction with a wound dressing after surgery) by injection, by means of a catheter, by means of a suppository, or by means of an implant (the implant being of a porous, non-porous, or gelatinous material, including membranes, such as sialastic membranes, or fibers). Preferably, when administering a protein, including an antibody, of the present invention, care must be taken to use materials to which the protein does not absorb.

In another embodiment, a biological material, such as a cell, a polypeptide, a polynucleotide, a compound or the like, or a composition thereof can be delivered in a vesicle, in particular a liposome (see Langer, Science 249: 1527-1533 (1990); Treat et al., Liposomes in the Therapy of Infectious Disease and Cancer, Lopez-Berestein and Fidler (eds.), Liss, N.Y., pp. 353-365 (1989); and Lopez-Berestein, supra, pp. 317-327).

In yet another embodiment, a biological material, such as a cell, a polypeptide, a polynucleotide, a compound or the like, or a composition thereof can be delivered in a controlled release system. In one embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105.(1989); Goodson, Medical Applications of Controlled Release, (Vol II, Chpt. 6. Boca Raton, Fla.: CRC Press, 1984.), Vol. 2, pp. 115-138 (1984); and Langer, Science 249:1527-1533 (1990)).

The amount of a cell, a polypeptide, a polynucleotide, or a compound used in the method of the present invention can be easily determined by those skilled in the art with reference to the purpose of use, a target disease (type, severity, and the like), the patient's age, weight, sex, and case history, the form or type of the cell, and the like. The frequency of the method of the present invention applied to a subject (or patient) is also determined by those skilled in the art with respect to the purpose of use, target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

As used herein, the dose of a biological material, such as a polypeptide, a polynucleotide or the like, varies depending on the subject's age, weight and condition or an administration method, or the like, including, but not limited to, ordinarily 0.01 mg to 10 g per day for an adult, preferably 0.1 mg to 100 mg, 0.1 mg to 100 mg, 1 mg to 100 mg, 0.1 mg to 10 mg, and the like. The present invention is not so limited.

In the present invention, when a cell is administered, the amount of the cell to be administered varies depending on the cell type, the purpose of treatment, a subject's age, weight and condition, or the administration method, and may be, but not particularly limited to, about 1×10³ cells to about 1×10⁸ cells per day for an adult, preferably about 1×10⁴ bells to about 1×10⁷ cells, and the like. Therefore, the amount administered one time may be between, for example, about 1×10³ cells to 1×10⁸ cells.

As used herein, the terms “administer” or “inject” are used interchangeably and each means that the biological material, such as a polypeptide, a polynucleotide, a compound, a cell, or the like, of the present invention or a pharmaceutical composition containing it is incorporated into cells or tissues of organisms either alone or in combination with other therapeutic agents. Combinations may be administered either concomitantly (e.g., as an admixture), separately but simultaneously or concurrently; or sequentially. This includes presentations in which the combined agents are administered together as a therapeutic mixture, and also procedures in which the combined agents are administered separately but simultaneously (e.g., as through separate intravenous lines into the same individual). “Combination” administration further includes the separate administration of one of the compounds or agents given first, followed by the second.

As used herein, the term “instructions” describe a method of administering a medicament, a method for diagnosis, or the like of the present invention for persons who administer, or are administered, the medicament or the like or persons who diagnose or are diagnosed (e.g, physicians, patients, and the like). The instructions describe a statement indicating an appropriate method for administering a diagnostic, a medicament, or the like of the present invention. The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions are so-called package insert and are typically provided in paper media. The instructions are not so limited and may be provided in the form of electronic media (e.g., web sites, electronic mails, and, the like provided on the Internet).

The judgment of termination of treatment with a method of the present invention may be supported by a result of a standard clinical laboratory using commercially available assays or instruments or extinction of a clinical symptom characteristic to a disease relevant to intended treatment (e.g., gene therapy, implantation therapy, regeneration therapy, cell implantation therapy, or the like).

The present invention also provides a pharmaceutical package or kit comprising one or more containers filled with one or more pharmaceutical compositions. A notice in a form defined by a government agency which regulates the production, use or sale of pharmaceutical products or biological products may be arbitrarily attached to such a container, representing the approval of the government agency relating to production, use or sale with respect to administration to humans. The kit may comprise an injecting device.

Toxicity studies may be carried out by measuring an influence of the administration of a composition. For example, a toxicity study may be carried out in the following appropriate animal model: (1) a compound is administered into mice (an untreated control mouse should also be used); (2) a blood sample is periodically obtained from a mouse in each treatment group via the tail vein; and (3) the sample is analyzed for the numbers of erythrocytes and leukocytes, the blood cell composition, and the ratio of lymphocytes and polymorphonuclear cells. Comparison of the result of each drug regimen with the control shows whether or not toxicity is present.

At the end of each toxicity study, a further study may be carried out by sacrificing the animal (preferably, in accordance with American Veterinary Medical Association guidelines Report of the American Veterinary Medical Assoc. Panel on Euthanasia, (1993) J. Am. Vet. Med. Assoc. 202: 229-249). Thereafter, a representative animal from each treatment group may be tested by viewing the whole body for direct evidence of transitions, abnormal diseases or toxicity. A global abnormality in tissue is described and the tissue is hisotologically tested. A compound causing a reduction in weight or a reduction in blood components is not preferable as are compounds having an adverse action in major organs. In general, the greater the adverse action, the less preferable the compound.

(Gene Therapy)

In a specific embodiment, the present invention is useful for administration of a nucleic acid for use in gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al.; Clinical Pharmacy 12: 488-505 (1993); Wu and Wu, Biotherapy 3: 87-95 (1991); Tolstoshev, Ann. Rev. Pharmacol. Toxicol. 32: 573-596 (1-993): Mulligan, Science 260: 926-932 (1993); and Morgan and Anderson, Ann. Rev. Biochem. 62: 191-217 (1993): May, TIBTECH 11 (5): 155-215 (1993). Methods commonly known in the art of recombinant DNA technology which can be used in gene therapy are described in Ausubel et al. (eds.), Current Protocols in Molecular Biology, John Wiley & Sons, N.Y. (1993); and Kriegler, Gene Transfer and Expression, A Laboratory Manual, Stockton Press, N.Y. (1990).

Therefore, gene therapy using a nucleic acid encoding a therapeutic protein or the like for achieving a desired purpose may be useful in the present invention.

(Injector)

As used herein, the term “injector” refers to a means for injecting a biological material into organisms, including, but not limited to, a syringe, a catheter, a needle, a tube, an endoscope, and the like. Therefore, an injector for use in the device of the present invention may be made of any material and in any shape as long as it can be used to inject a biological material into organisms. As a material for the injector, any solid material which does not have an adverse influence on an organism subjected to injection, or a solid material coated with a material which does not have an adverse influence on an organism subjected to injection, is illustrated. Therefore, examples of such an injector material include, but are not limited to, any material capable of forming a solid surface, such as, glass, silica, silicone, polytetrafluoroethylene (PTFE), ceramics, silicon dioxide, plastics, metals (including alloys), natural and synthetic polymers (e.g., biodegradable, polymers (e.g., PGA, PLGA, PLA, PCLA, etc.),polystyrene, cellulose, chitosan, dextran, and nylon), sugars, proteins, lipids, and the like. The injector may be formed of a plurality of different materials. The biocompatibility of the injector can be confirmed by investigating a rejection reaction with, for example, biochemical quantitation (e.g., SDS-PAGE, a labeled collagen method, etc.), immunological quantitation (e.g., an enzymatic antibody method, a fluorescent antibody method, immunohistological study, etc.). Alternatively, an injector for use in the present invention may contain a component which can become a part of an organism. Examples of such a component include, but are not limited to, silicone, ceramics, proteins, lipids, nucleic acids, sugars (carbohydrates) and complexes thereof. Any injector that maintains a flow rate within a predetermined range can be used in the present invention, since the effect of the present invention can be achieved by such an injector. If an injector of the present invention is in the shape of a syringe, a catheter, a needle, a tube, or the like, an inner diameter of the body of the injector having such a shape is typically about 1 mm to about 30 mm. Note that it was demonstrated that the effect of the present invention (e.g., maintenance of the survival rate of cells, etc.) can be achieved by controlling only the flow rate irrespective of the inner diameter of the body of the injector. The inner diameter of the body of an injector may be advantageously, but is not limited to, about 3 mm (e.g., about a 0.5-ml syringe) to about 13 mm (e.g., about a 5.0 ml syringe) in terms of handling of the injector. An inner diameter of a tip tube of the injector is not particularly limited, and may be preferably about 0.1 mm to about 10 m, more preferably about 0.25 mm to 0.5 mm. Note that it was demonstrated that the effect of the present invention (e.g., maintenance of the survival rate of cells, etc.) can be achieved by controlling only the flow rate irrespective of the inner diameter of the tip tube of the injector. A material of the injector and the tip tube may be preferably, but is not limited to, PTFE.

As used herein, the term “tip tube” in relation to an injector refers to a means for injecting a liquid for injection within the body of the injector, which can be connected to a tip of the body and can be connected to a subject into which the liquid is to be injected. Examples of a tip tube include, but are not limited to, needle catheters, tubes, and the like. A tip tube typically constitutes a part of an injector.

As used herein, the term “body” in relation to an injector refers to a main part of the injector capable of retaining a liquid drug. An example of such a body includes, but is not limited to, a syringe body and the like.

A support used herein may be made of any material from which a component toxic to a targeted subject is not dissolved into a solution containing a biological material.

As used herein, the term “coated” in relation to a support refers to a state of the support in which the support is covered with another material. Therefore, a support can be coated with a material which can interact with the support. A support may be coated so that a material for the support itself does not contact with the outside (e.g., the air). If a support and a coating material can interact with each other to a certain degree, the support may not be coated to the extent that the support can contact with the outside. The degree of the coating is arbitrarily determined and can be adjusted by those skilled in the art using a well-known technique in the art. An exemplary coating technique is described in, for example, “Kobunshi Kino Zairyo Sirizu Iryo Kobunshi Kino Zairyo [Medical functional Materials in the Course of Polymer Functional Materials”, Kyoritsu Shuppan K.K.

As used herein, the term “velocity” refers to an injection velocity of a liquid drug containing a cell unless otherwise specified. In the present invention, the importance of maintaining a predetermined flow rate unchanged and the effect thereof have been unexpectedly found. The flow rate can be represented by linear velocity. Therefore, it will be understood that the velocity may be represented by linear velocity if particularly specified, though the velocity usually means a flow rate.

As used herein, an “acceleration” is represented by a linear acceleration unless otherwise specified. The relationship between a velocity and an acceleration is described in detail in other portions of the specification, and will be understood by those skilled in the art.

As used herein, the term “adjustor” in relation to an injection velocity and/or acceleration refers to a means which may be connected to an injector and is used for adjusting the injection velocity and/or acceleration of a liquid drug or the like of the injector. An injector preferable for a system of the present invention is constructed so that a liquid drug containing a biological material may be accelerated within a predetermined acceleration range and the injection velocity of the liquid drug containing the biological material may be maintained within a predetermined range. The acceleration and velocity can be adjusted by a combination of techniques well-known in the art. Such an adjustment can be achieved by a device, including, but not limited to, a microinjector, a peristatic pump, and the like. Well-known techniques as described above are described in, for example, Seimei Kogaku Sirizu 1 [Biotechnology Series 1], Seimei Kikai Kogaku [Biomechanics], Takayuki Miwa, editor, Shokabo, 1992. The above-described publications are herein incorporated by reference in their entity.

The above-described acceleration and velocity can be adjusted manually, preferably automatically. With automatic control, it is possible to suppress a sudden change in velocity and/or acceleration which is likely to occur in manual control. Such automatic control can be preferably achieved by, for example, a liquid drug injecting device of the present invention, or other controllable devices.

(Description of the Preferred Embodiments)

Hereinafter, preferred embodiments of the present invention will be described. The following embodiments are provided for a better understanding of the present invention and the scope of the present invention should not be limited to the following description. It will be clearly appreciated by those skilled in the art that variations and modifications can be made without departing from the scope of the present invention with reference to the specification.

According to one aspect of the present invention, the present invention provides a method for injecting a liquid drug containing a biological material. The method comprises: A) injecting the liquid drug containing the biological material into an organism of interest while accelerating the liquid drug at a predetermined range of acceleration; and B) after a velocity of the liquid drug reaches a predetermined range of velocity, continuing the injection of the liquid drug containing the biological material while maintaining the velocity within the predetermined range of velocity.

Any material may be available for the biological material as long as it has compatibility to the organism, since an object of the present invention is to administer a liquid drug to organisms. Preferably, the biological material may be advantageously one having a medical effect (e.g., a therapeutic effect, a prophylactic effect, a prognostic effect, etc. In one embodiment, the biological material may contain at least one selected from the group consisting of cells, polypeptides, and polynucleotides. Particularly, in conventional method for injecting cells into organisms, there are problems with the survivability of the cells and precious cell resources, such as stem cells or the like, cannot be effectively utilized. In the present invention, cells can be injected into organisms without impairing the survivability of the cells. Therefore, this advantageous effect cannot be achieved by conventional techniques. The effect of an increase in the effectiveness of injection into organisms can be obtained by the present invention for other biological materials (e.g., polypeptides, polynucleotides, and other drugs). Thus, the present invention is useful for general biological materials.

In the present invention, the acceleration is preferably in a range in which the biological material is not damaged and the injection time is not significantly elongated. The upper limit of the acceleration which does not damage the biological material may be, for example, 30 mm/sec², preferably 20 mm/sec², more preferably 15 mm/sec², and most preferably 10 mm/sec². The upper limit value may vary depending on the biological material to be injected, and can be determined by those skilled in the art if required. The lower limit value may be determined by those skilled in the art if required. The lower limit value may be, for example, about 0.1 mm/sec², preferably.0.5 mm/sec², more preferably 1 mm/sec², and even more preferably 5 mm/sec². Therefore, a preferable predetermined range of acceleration may be any combination of lower limit values and upper limit values as described above. An exemplary predetermined range of acceleration may be 1 to 15 mm/sec².

In the present invention, the velocity may be preferably within a predetermined range. Particularly, the upper limit of the velocity is a level at which a biological material, such as a cell, is not damaged. Conventionally, the velocity of a cell being injected has not been studied. Therefore, when a biological material is injected manually, the effectiveness of the material is significantly reduced. The present invention overcomes such disadvantages of conventional techniques, so that a biological material having problems with maintenance of the effectiveness, such as a cell, can be injected into organisms while retaining the effectiveness. In addition, as illustratively demonstrated in the Examples below, a significant therapeutic or prophylactic effect was achieved in the present invention. Furthermore, by injecting a biological material such as a cell at a predetermined range of velocity (in this case, flow rate), the cell can be introduced into organisms without substantial damage to the cell, independent of the diameter, volume, or the like of a syringe. Such independence is unexpected in terms of physical common sense where parameters, such as a pressure and the like, are taken into consideration. In this point, the present invention can be said to have an unexpected effect.

In a certain embodiment of the present invention, the upper limit of the velocity (flow rate) may be preferably 20 ml/min, 15 ml/min, more preferably less than about 10 ml/min, and even more preferably about 8 ml/min. The lower limit value of the velocity is also preferably determined to be greater than or equal to a certain value by considering the amount of injection to an organism. Such a lower limit value can be determined by those skilled in the art if required. The lower limit of a predetermined range of velocity is preferably 0.1 ml/min, for example. The velocity range may be preferably from about 1 ml/min to about 10 ml/min. The present invention is not limited to this. In the present invention, it is necessary to determine the lower and upper limits of the flow rate by using a syringe pump. As a factor for determining the lower limit, for example, it is believed that if adhesive cells which are not floating cells are suspended for a long time, the survival rate of the cells is adversely affected. Therefore ,the lower limit can be determined by those skilled in the art if required, taking into consideration the injection amount and the injection time. Thus, in the present invention, the determination of the upper limit of the injection velocity is the most important thing.

In a preferred embodiment of the present invention, a biological material to be injected includes a material selected from the group consisting of nucleic acid molecules, polypeptides, lipids, sugar chains, small organic molecules, and complex molecules thereof, cells, tissues, and organs. In one preferred embodiment, the biological material may be a nucleic acid molecule for use in gene therapy. In another preferred embodiment, the biological material may be a cell for use in regeneration therapy.

In a particular embodiment of the present invention in which a cell is injected, the acceleration is preferably in the range of 1 to 15 mm/sec², and the velocity is preferably in the range of about 1 ml/min to about 10 ml/min. The present invention is the first to achieve injection of a cell into organisms without significantly damaging the cell by maintaining the acceleration and/or the velocity within a predetermined range.

In a particular embodiment of the present invention in which a nucleic acid molecule for gene therapy is injected, the acceleration is in the range of 1 to 15 mm/sec², and the velocity is in the range of about 1 ml/min to about 10 ml/min. The present invention is the first to achieve injection of a cell into organisms without significantly impairing the expression of the nucleic acid molecule, which relates to a therapeutic or prophylactic effect, by maintaining the acceleration and/or the velocity within a predetermined range.

In another aspect of the present invention, a system for injecting a liquid drug containing a biological material is provided. The injecting system comprises A) an injector for injecting the liquid drug containing the biological material into an organism of interest while accelerating the liquid drug at a predetermined range of acceleration; and B) a continuer for continuing the injection of the liquid drug containing the biological material while maintaining a velocity of the liquid drug within a predetermined range of velocity after the velocity of the liquid drug reaches the predetermined range of velocity.

The injector is any means that can maintain a predetermined range of acceleration and/or a predetermined range of velocity. The injector can be fabricated by those skilled in the art using techniques well-known in the art. Alternatively, the injector may be obtained by combining commercially available means. Therefore, such means may be made of any material as long as the means can be employed to inject a biological material. Since an object of the present invention is to administer biological material into organisms, the injector is preferably made of a biocompatible material.

In order for the injector to maintain a predetermined range of acceleration, the upper limit of the acceleration range is particularly preferably limited. A sensor for detecting when the acceleration reaches the upper limit and a mechanism for suppressing the acceleration when the acceleration reaches the upper limit are preferably provided in order to maintain the acceleration within a predetermined range. As described above, in the method of the present invention, the predetermined range of acceleration is advantageously 1 to 15 mm/sec². Therefore, a means capable of maintaining such an acceleration is preferable. An acceleration can be determined with physical parameters, such as pressure and the like. A device for maintaining an acceleration within the above-described range includes the illustrated devices of the present invention and equivalents thereof.

In order for the injector to maintain a predetermined range of velocity, the upper limit of the velocity range has to be limited. Thus, a mechanism is provided for stopping the injection when the velocity reaches the upper limit. Therefore, a means for measuring the flow rate and a mechanism for limiting the flow rate when the flow rate reaches the upper limit are preferably provided in order to maintain the velocity within a predetermined range. As described above, in the method of the present invention, the predetermined range of velocity is advantageously about 1 ml/min to about 10 ml/min. Therefore, a means capable of maintaining such a velocity is preferable. A device for maintaining a velocity within the above-described range includes the illustrated devices of the present invention and equivalents thereof.

Examples of a biological material, which is injected using a device or system of the present invention, include, but are not limited to, a material selected from the group consisting of nucleic acid molecules, polypeptides, lipids, sugar chains, small organic molecules and complexes thereof, and cells, tissues, and organs. In the present invention, such a biological material could be efficiently injected into organisms without impairing the substantial effectiveness. This is significantly beneficial for regeneration medicine, implantation, and gene therapy. Particularly in the present invention, by limiting the injection velocity itself, cells could be injected into organisms without impairing the substantial survivability (e.g., cell proliferation activity) of the cells. Conventionally, this was unknown and is not even expected. Thus, the present invention has an unexpected effect.

In a preferred embodiment, an inner diameter of the body of an injector in the system of the present invention is typically about 1 mm to about 30 mm. Note that it was demonstrated that the effect of the present invention (e.g., maintenance of the survival rate of cells, etc.) can be achieved by controlling only the flow rate irrespective of the inner diameter of the body of the injector. The inner diameter of the body of an injector may be advantageously, but is not limited to, about 3 mm (e.g., about 0.5-ml syringe) to about 13 mm (e.g., about 5.0 ml syringe) in terms of handling of the injector. An inner diameter of a tip tube of the injector is not particularly limited, and may be preferably about 0.1 mm to about 10 mm, more preferably about 0.25 mm to 0.5 mm. Note that it was demonstrated that the effect of the present invention (e.g., maintenance of the survival rate of cells, etc.) can be achieved by controlling only the flow rate irrespective of the inner diameter of the tip tube of the injector.

(Description of Illustrative Mechanism)

The present invention is achieved by a system in which by controlling injection acceleration and injection velocity, cell implantation or gene introduction can be achieved without damaging the inside of a cell and a gene. The present invention also relates to such a system.

(Exemplary Liquid Drug Injecting Device of the Invention)

Hereinafter, the present invention will be described with reference to the accompanying drawings.

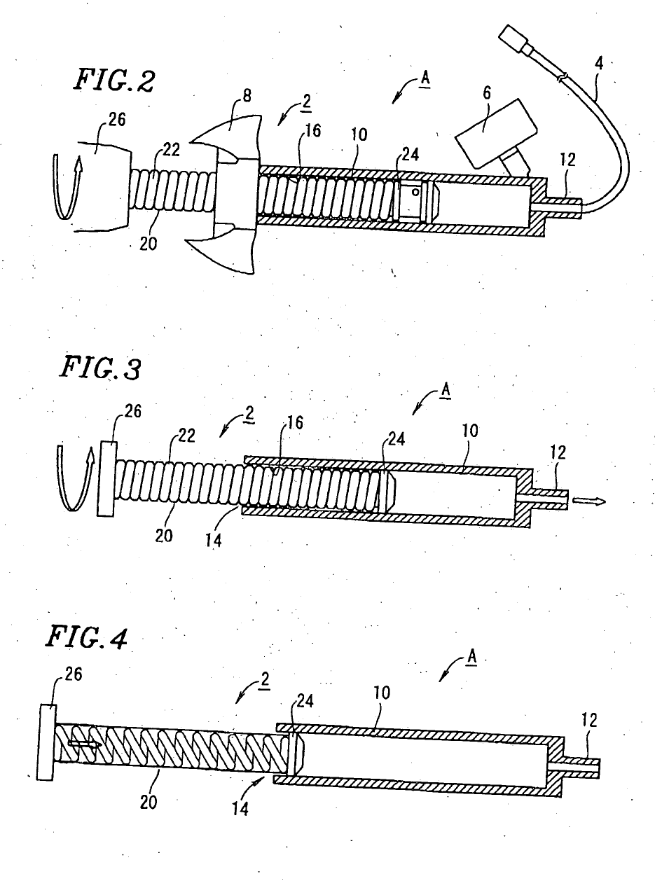

Referring to FIGS. 2 to 14, a liquid drug injecting device A can be filled with a liquid drug, and comprises: a nozzle portion 12 for outputting the liquid drug provided at a tip portion thereof; a cylinder 10 having an opening portion 14 provided at a rear end portion thereof; and pushing portion 2 for pushing out the liquid drug contained in the cylinder 10 through the nozzle portion 12 by external control while maintaining a predetermined velocity or acceleration substantially unchanged.

The cylinder 10 is in the shape of a cylinder and can be made of glass or a transparent plastic. The cylinder 10 is typically provided with scales indicating the volume of a loaded liquid drug.

In the example shown in FIGS. 2 and 3, the pushing portion 2 comprises a plunger 20 provided with a screw-thread portion 22 arranged around the outer perimeter thereof so that the plunger 20 can be moved into the cylinder 10, and a nut-thread portion 16 provided on an inner wall of the cylinder 10 so that the screw-thread portion 22 of the plunger 20 is engaged with the nut-thread portion 16. A plug 24 is provided at a tip portion of the plunger 20 so that the plug 24 contacts and presses the inner wall of the cylinder 10 in a watertight manner. A handle portion 26 is provided at a rear end portion of the plunger 20. By holding the handle portion 26 and rotating the plunger 20, the screw-thread portion 22 of the plunger 20 is engaged with the nut-thread portion 16 on the inner wall of the cylinder 10 so that the plunger 20 is moved forward along the cylinder 10. In this case, the plug 24 pushes out the liquid drug contained in the cylinder 10 through the nozzle portion 12.

The nozzle portion 12 is connected to a tube 4 through which the liquid drug is introduced into the body.

A velocity or acceleration measuring instrument (or pressure gauge) 6 for measuring the velocity or acceleration of the liquid drug is attached to a tip portion of the cylinder 10 and is provided in communication with the cylinder 10. A releasing portion 8 (e.g., a handle, etc.) is provided at a rear end portion of the cylinder 10 for releasing the engagement of the screw-thread portion 22 of the plunger 20 and the nut-thread portion 16 of the cylinder 10. The screw-thread portion 22 of the plunger 20 is engaged with the nut-thread portion 16 of the cylinder 10 by rotating the releasing portion 8 clockwise. The engagement can be released by rotating the releasing portion 8 counter-clockwise. The nut-thread portion 16 of the cylinder 10 is typically provided only at a portion of the cylinder 10, such as a rear portion of the inner wall of the cylinder 10.

A preferred example of a liquid drug for use in the present invention includes, but is not limited to, liquid containing cells.

In a liquid drug injecting device A shown in FIG. 4, a pushing portion 2 comprises a plunger 20 which can be moved into the cylinder 10, and a plug 24 provided at a tip portion of the plunger 20. The plunger 20 comprises a spring-like elastic member which can be compressed when the velocity or acceleration is greater than or equal to a predetermined value. The spring-like elastic member can be made of a metal, a resin, a rubber, a gel, or the like. A handle portion 26 (e.g., a flange, etc.) is provided at a rear end portion of the plunger 20.

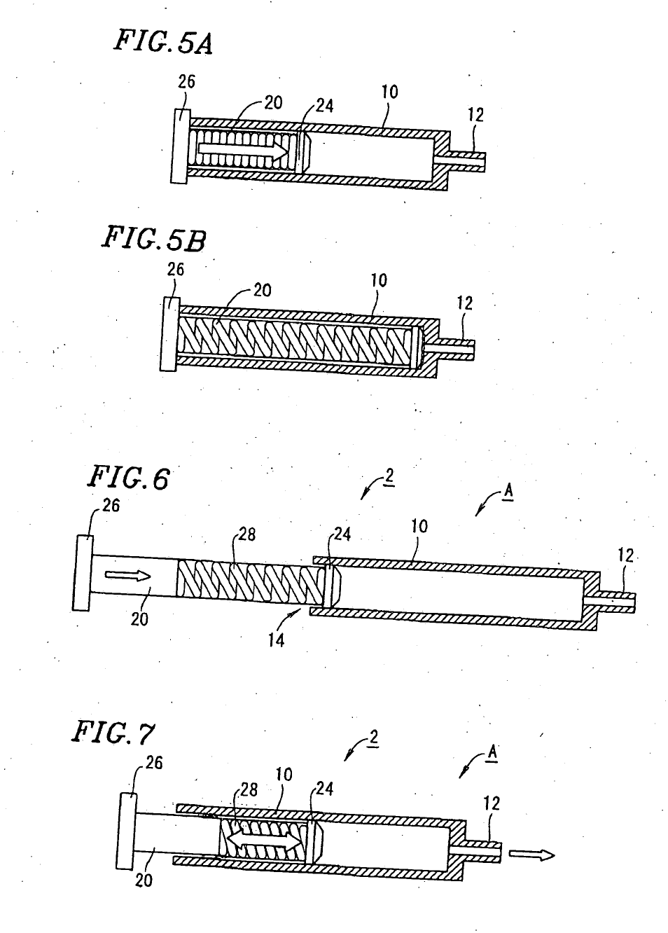

It is assumed that the plunger 20 is manually pushed into the cylinder 10 in a rapid manner. Even in this case, the velocity or acceleration of the liquid drug contained in the cylinder 10 can be prevented from becoming greater than or equal to a predetermined value. This is because when the velocity or acceleration of the plunger 20 is greater than or equal to a predetermine value defined by the spring modulus, a predetermined force or more is exerted onto the plunger 20, so that the plunger 20 is compressed as shown in FIG. 5A. The plunger 20 is pushed with a predetermined force corresponding to the spring modulus of the plunger 20 so as to inject the liquid drug through the nozzle portion 12 into the body as shown in FIG. 5B.

In a liquid drug injecting device A shown in FIGS. 6 and 7, a pushing portion 2 comprises a plunger 20 provided in a cylinder 10, and an elastic member 28 provided at a tip portion of the plunger 20. In this example, the elastic member 28 is compressed when the velocity or acceleration thereof is greater than or equal to a predetermined value. Also in this example, a liquid drug is prevented from being injected in an excessive amount due to manual operation by the compression of the elastic member 28. A plug 24 is provided at a tip portion of the elastic member 28.

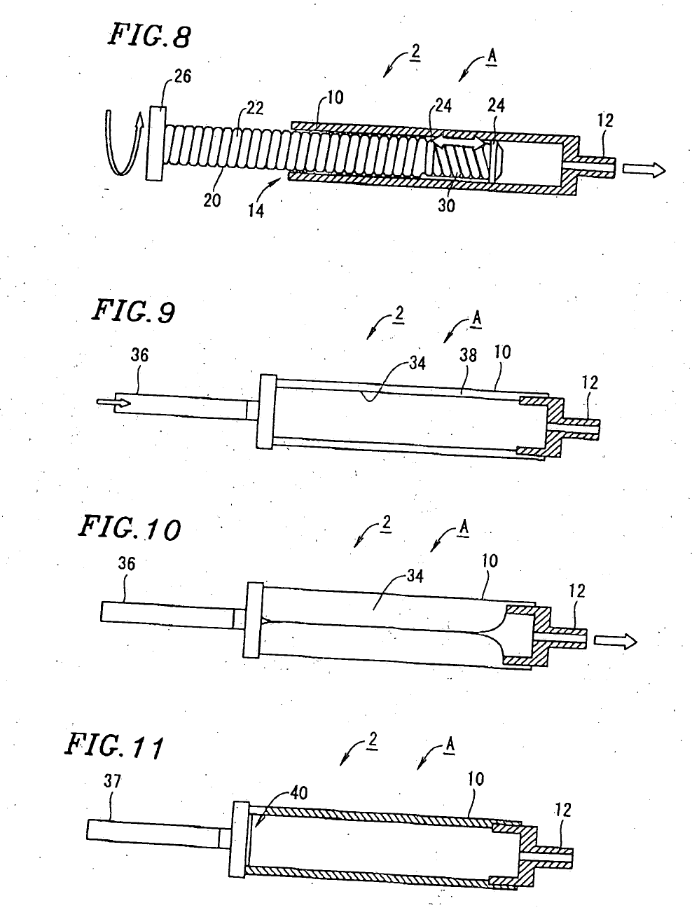

In a liquid drug injecting device A shown in FIG. 8, a pushing portion 2 comprises a plunger 20 provided with a screw-thread portion 22 on the outer perimeter thereof so that the plunger 20, can be moved into a cylinder 10, a nut-thread portion 16 provided on an inner wall of the cylinder 10 so that the screw-thread portion 22 of the plunger 20 is engaged therewith, and an elastic member 30 provided at a tip portion of the plunger 20. When the velocity or acceleration of the plunger 20 is greater than or equal to a predetermined value, the elastic member 30 is compressed. A plug 24 is provided at a tip portion of the elastic member 30.

By holding a flange and a rotating the plunger 20, the screw-thread portion 22 is engaged with the nut-thread portion 16, so that the plunger 20 is moved forward through the cylinder 10. With this operation, a sudden or rapid injection manipulation can be prevented. In addition, since the elastic member 30 is compressed in accordance with the spring modulus thereof, a liquid drug can be injected at a constant velocity or acceleration with a predetermined pushing force.

In a liquid drug injecting device A shown in FIG. 9, a pushing portion 2 comprises an inflating member 34 provided on the inner perimeter surface of the cylinder 10, and a loading portion 36 for loading an incompressible fluid into the inflating member 34. The inflating member 34 can be made of a rubber, an elastomer, or the like, and is in the shape of a cylinder, extending over the entire inner perimeter surface of the cylinder 10. A spacing portion 38 is formed between the inflating member 34 and the cylinder 10. A flow path is provided at a rear end portion of the cylinder 10 for supplying the incompressible fluid from a source to the spacing portion 38 (not shown). As shown in FIG. 10, when the incompressible fluid is loaded into the spacing portion 38 at a substantially constant velocity or acceleration by the loading portion 36, the volume of the cylinder 10 is reduced so that the liquid drug is pushed out through the nozzle portion 12 at a constant velocity or acceleration.

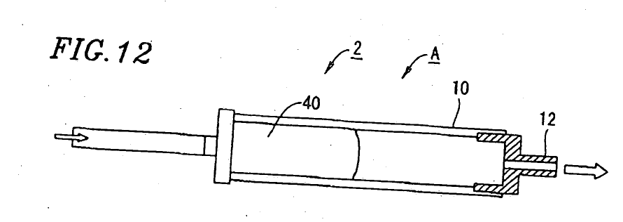

In a liquid drug injecting device A of FIG. 11, a pushing portion 2 comprises a hollow inflating member 40 attached to a rear end portion of the cylinder 10, and a loading portion 37 for loading an incompressible fluid into the inflating member 40. As shown in FIG. 12, the incompressible fluid is loaded into the inflating member 40 by the loading portion 37 at a substantially constant velocity or acceleration, so that the volume of the cylinder 10 is reduced and the liquid drug is pushed out through the nozzle portion 12.

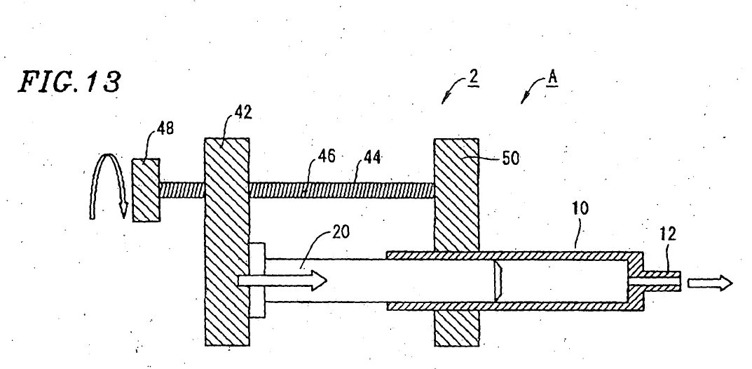

In a liquid drug injecting device A shown in FIG. 13, a pushing portion 2 comprises a plunger 20 movably attached to a cylinder 10, and a driving portion for inserting the plunger 20 into the cylinder 10 at a constant velocity.

In this example, the driving portion comprises a plate 42 fixed at a rear end portion of the plunger 20, a rotatory shaft 46, and a flange 48 for rotating the rotatory shaft 46. The rotatory shaft 46 has a male thread 44 engaged with a female thread provided in the plate 42. A tip of the rotatory shaft 46 is supported by a base plate 50 fixed to the cylinder 10.

By rotating the flange 48 manually, the rotatory shaft 46 is rotated so that the plate 42 and the plunger 20 are moved. As a result, a liquid drug contained in the cylinder 10 is pushed out through the nozzle portion 12 at a constant velocity or acceleration.

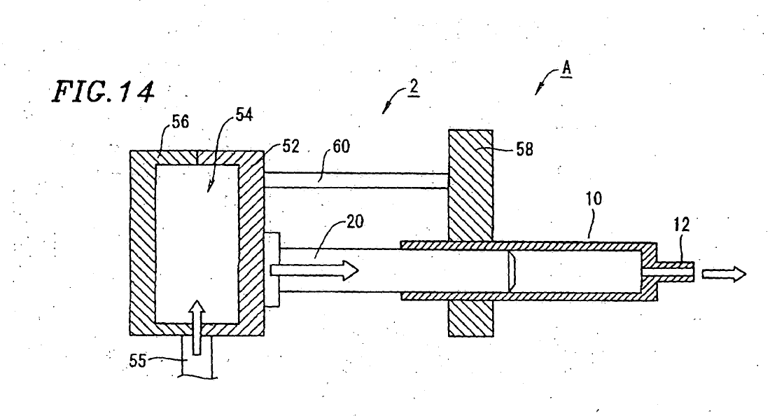

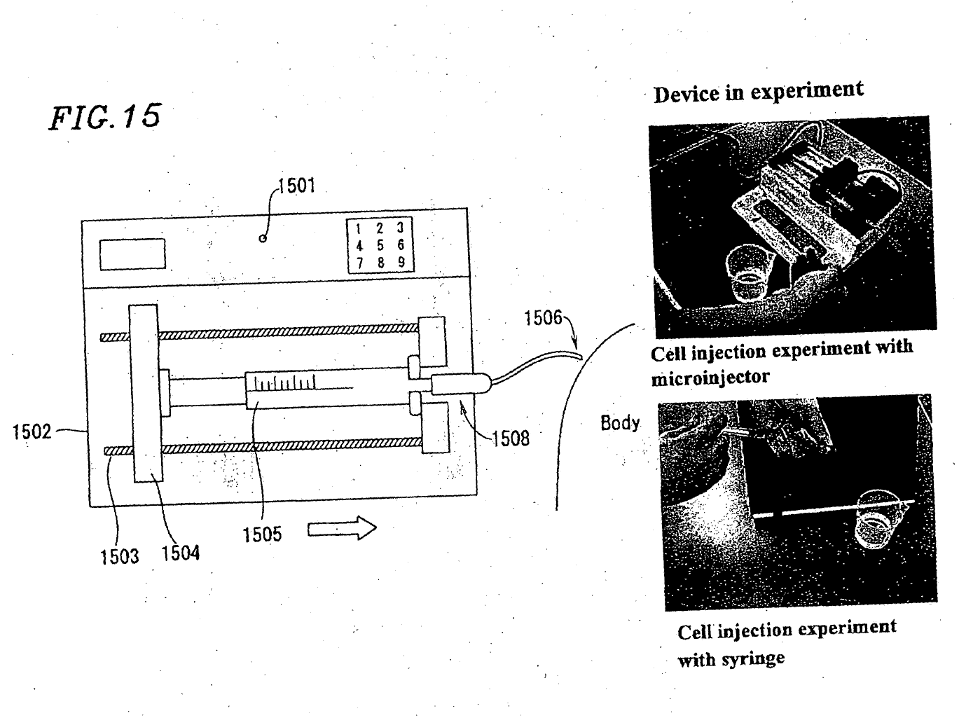

In an example shown in FIG. 14, a driving portion comprises a movable portion 52 fixed at a rear end portion of a plunger 20, a fixing portion 56 which forms a cavity 54 together with the movable portion 52, and a loading portion 55 for loading an incompressible fluid into the cavity 54. The movable portion 52 is slidably supported via a rod 60 by the base plate 58 fixed to the cylinder 10.

When an incompressible fluid is loaded through the loading portion 55 into the cavity 54, the movable portion 52 and the plunger 20 are moved, so that the liquid drug contained in the cylinder 10 is pushed out through a nozzle portion 12 at a constant velocity or acceleration.

(Configuration of System of the Invention)

An exemplary configuration of a concrete system will be described below. A system for injecting a liquid drug containing a cell, a polypeptide, a gene, or the like according to the present invention may comprise:
(1) a plunger pushing device to which various syringes are attached and which can control the operation of the syringe plunger in accordance with a program; and
(2) a micro-catheter which can be attached to the syringe and which comprises a rigid and flexible shaft with a needle at a tip thereof, where the micro-catheter is to be inserted into the body.

An exemplary configuration is shown in FIG. 15. Referring to FIG. 15, the system comprises an injection control panel 1501, a syringe fixing portion 1502, a syringe 1505, an injection portion 1508, a micro-catheter 1506 with a needle, and the like. In the injecting-device, a plate 1504 is moved by a shaft 1503 with threads being rotated by control of the control panel so as to push the syringe. The syringe can be fixed with a clamp.

(Exemplary Program for a System of the Invention Upon Injection)

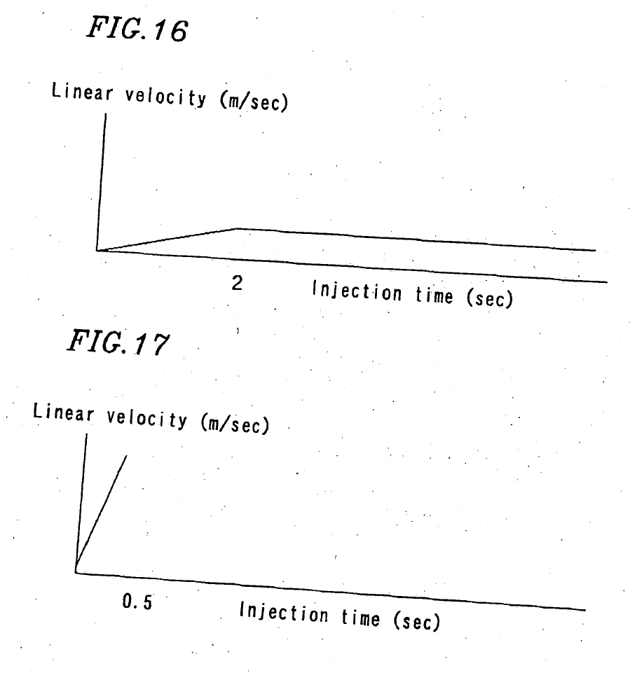

A program for a system of the present invention which is used upon injection will be described. Here, the linear velocity (v) of al syringe plunger when injection is being performed is represented in the unit m/sec and the injection time is represented in the unit seconds. A 1-ml syringe was used under the condition (flow rate: 25 ml/min). The injection time and linear velocity of a system of the present invention are shown in FIG. 16.

In this case, it takes two seconds for the flow rate to reach 25 ml/min, i.e., for the velocity of the syringe plunger to reach 0.02 m/sec. The acceleration a (m/sec²) of the syringe plunger is calculated by dv/dt to be 0.01 m/sec². Thereafter, the syringe plunger is in uniform motion, where no acceleration is generated. By changing the elapsed time required for the flow rate to reach a predetermined value, the acceleration of the syringe plunger can be maintained less than or equal to a certain value, where the syringe plunger is subsequently in uniform motion and no acceleration is generated. In the system, the relationship between the predetermined flow rate (m³)/min), and the acceleration a (m/sec²) of the syringe plunger and the elapsed time t (sec) required for the flow rate to the predetermined value is represented by:
Predetermined flow rate (m³/min)=S(m²)·10⁶·a·t·60
where the cross-sectional area of the syringe is represented by S (m²).

According to the above-described relational expression, the acceleration of the syringe plunger in this experiment is 0.001 to 0.015 m/sec². When the program for the system upon injection is used, the proliferation ability of cells is not reduced. It can be thus said that cells are not damaged. In addition, by controlling the acceleration so as to prevent the rapid rise of the flow rate, cell implantation or gene introduction can be carried out without damaging the inside of a cell and a gene.

The injection time and linear velocity of a 1-ml syringe where the syringe is manually moved and the flow rate is 38 ml/min will be shown in FIG. 17.

It takes 0.5 sec for the flow rate to reach a predetermined value 38 ml/min, i.e., for the linear velocity of the plunger to reach 0.05 m/sec. Here, the acceleration is calculated by dv/dt to be 0.1 m/sec². In this case, it is considered that the manual injection having an acceleration of 0.1 m/sec² damages cells so that the proliferation ability of the cells after injection is suppressed. Further, the high acceleration causes the rapid rise of the flow rate, leading to damage to the inside of a cell and a gene.

(Illustrative Program flow of a System of the Invention upon Injection)

(1) Input initial settings: a flow rate value, the inner diameter of a syringe, an elapsed time t (sec).

(2) Start the system using a start button.

(3) Start a timer.

(4) Automatically calculate a target syringe plunger acceleration a (m/sec²); when the acceleration exceeds an upper limit acceleration, outputs an error message and return to (1).

(5) Start the movement of a syringe plunger.

(6) Measure the moving distance of the syringe plunger and obtain the acceleration of the syringe plunger based on the moving distance and the time indicated by the timer using a second order differentiation circuit. If the acceleration is greater than the upper limit, reduce the velocity of the movement (5). If the acceleration is less than the upper limit, increase the velocity of the movement (5).

(7) Measure the moving distance of the syringe plunger at the elapsed time t (sec) indicated by the timer, and calculate the flow rate based on the moving distance and the time indicated by the timer using a first-order differentiation circuit. If the deviation from the initial set value is within ±5%, continue movement of the syringe plunger. If the deviation exceeds ±5%, output an error message and return to (1).

(8) Measure the moving distance of the syringe plunger. If detecting the dead end, output a stop message and return to (1).

Hereinafter, the present invention will be described by way of examples. Examples described below are provided only for illustrative purposes. Accordingly, the scope of the present invention is not limited except as by the appended claims.

### (EXAMPLES)

The present invention will be described in greater detail by way of examples. The present invention is not limited to the examples below. Animals were treated in accordance with rules defined by Osaka University (Japan).

(Example 1)

A pressure drop caused when a liquid passes through a small tube is considered to be significantly affected by the viscosity of the liquid, the type of cell, the length of the device, and the size of syringe. In this example, in order to investigate a pressure drop during injection, a liquid drug containing a dummy cell and a syringe (injector) were employed to perform injection experiments under various conditions.

Specifically, devices shown in Table 1 and FIG. 1 were used. The syringes were operated with a system or manually. A cell-containing liquid drug was injected under various conditions shown in Table 2. For the manual injection using the syringe, the acceleration was measured before experiments and was found to be 0.1 m/sec².

(Preparation of Cells)

Rat skeletal myoblasts (primary culture cells) were used as samples. The cells were maintained in a medium (DMEM (High Glucose) (Gibco), supplemented with fetal bovine serum (FBS) (Gibco), 2 mM L-glutamine, 50 units/mL penicillin and 50 μg/mL streptomycin) in 5% CO₂ at 37° C.

(Measurement)

A cell-containing liquid drug was collected after injection. The instantaneous cell activity was investigated by a trypan blue staining method and the cell activity was investigated over time by an MTT method. Cell-containing liquid drug samples were injected using the above-described sample devices. Changes in cells immediately after injection and over time were investigated by changing the length of the device, the injection velocity, and the size of the syringe. Hereinafter, the trypan blue staining method and the MTT method will be described.

Trypan Blue Staining Method

Trypan blue was dropped into a cell-containing liquid drug immediately after injection. A cell stained by a trypan blue dye pigment was determined as a dead cell. Trypan blue is one of several stains recommended for use in dye exclusion procedures for viable cell counting. This method is based on the principle that live (viable) cells do not take up certain dyes, whereas dead (non-viable) cells do.

After trypan blue staining, cells were counted and assessed with a microscope.

MTT Method

This is a method for assessing cell activity by utilizing the fact that tetrazolium salt is reduced to formazan by dehydrogenase of mitchondria in cells. The amount of produced formazan corresponds well to the number of cells. Formazan has an absorption characteristic to a particular wavelength. Therefore, the number of surviving cells can be easily determined by measuring the absorbance of a sample. In additions since the metabolism activity of mitochondria in cells is measured, cell death can be detected relatively early.

TABLE 1

| | Shaft | Shaft | | | | |
|---|---|---|---|---|---|---|
| | (tube) | (tube) | Shaft | | Needle | |
| | Mate- | Inner | (tube) | Needle | Inner | Needle |
| No. | rial | diameter | Length | Material | diameter | Length |
| Device I | PTFE | φ0.5 mm | 1350 mm | Ni—Ti | φ0.3 mm | 15 mm |
| Device | PTFE | φ0.5 mm | 1350 mm | Ni—Ti | φ0.3 mm | 40 mm |
| II | | | | | | |

Outline of Devices I, II
Inner- and Inter-layers: PTFE coating
SUS φ0.025 mm 4-line coil braiding
NiTi needle provided at a tip of a polyimide tube

Inner diameter: φ0.47 mm: Outer diameter: φ0.62 mm

TABLE 2

| No. | Injection velocity | Device | Syringe size |
|---|---|---|---|
| 1 | 1 ml/min (system) | I | 3-ml syringe |
| 2 | 1 ml/min (system) | I | 5-ml syringe |
| 3 | 3 ml/min (system) | I | 3-ml syringe |
| 4 | 3 ml/min (system) | I | 5-ml syringe |
| 5 | 5 ml/min (system) | I | 3-ml syringe |
| 6 | 5 ml/min (system) | I | 5-ml syringe |
| 7 | 9 ml/min (system) | I | 3-ml syringe |
| 8 | 9 ml/min (system) | I | 5-ml syringe |
| 9 | 12 ml/min (system) | I | 3-ml syringe |
| 10 | 12 ml/min (system) | I | 5-ml syringe |
| 11 | 15 ml/min (system) | I | 3-ml syringe |
| 12 | 15 ml/min (system) | I | 5-ml syringe |
| 13 | 20 ml/min (system) | I | 3-ml syringe |
| 14 | 20 ml/min (system) | I | 5-ml syringe |
| 15 | 25 ml/min (system) | I | 3-ml syringe |
| 16 | 25 ml/min (system) | I | 5-ml syringe |
| 17 | about 5 ml/min (manual injection | I | 1-ml syringe |
| | with syringe) | | |
| 18 | about 9 ml/min (manual injection | I | 1-ml syringe |
| | with syringe) | | |
| 19 | about 12 ml/min (manual injection | I | 1-ml syringe |
| | with syringe) | | |
| 20 | about 15 ml/min (manual injection | I | 1-ml syringe |
| | with syringe) | | |
| 21 | about 20 ml/min (manual injection | I | 1-ml syringe |
| | with syringe) | | |
| 22 | about 38 ml/min (manual injection | I | 1-ml syringe |
| | with syringe) | | |
| 23 | 5 ml/min (system) | II | 3-ml syringe |
| 24 | 5 ml/min (system) | II | 5-ml syringe |
| 25 | 9 ml/min (system) | II | 3-ml syringe |
| 26 | 9 ml/min (system) | II | 5-ml syringe |
| 27 | 12 ml/min (system) | II | 3-ml syringe |
| 28 | 12 ml/min (system) | II | 5-ml syringe |
| 29 | 20 ml/min (system) | II | 3-ml syringe |
| 30 | 20 ml/min (system) | II | 5-ml syringe |
| 31 | about 9 ml/min (manual injection | I | 1-ml syringe |
| | with syringe) | | |
| 32 | about 12 ml/min (manual injection | I | 1-ml syringe |
| | with syringe) | | |
| 33 | about 15 ml/min (manual injection | II | 1-ml syringe |
| | with syringe) | | |
| 34 | about 20 ml/min (manual injection | II | 1-ml syringe |
| | with syringe) | | |
| 35 | about 38 ml/min (manual injection | II | 1-ml syringe |
| | with syringe) | | |
| 36 | control (no injection operation) | | |

The results of a trypan blue staining method for detecting cell death immediately after an injection operation are shown in Table 3.

TABLE 3

| | Living cell | Dead cell concentration |
|---|---|---|
| No. | concentration (cells/ml) | (cells/ml) |
| 1 | 460000 | 0 |
| 2 | 450000 | 0 |
| 3 | 480000 | 0 |
| 4 | 450000 | 0 |
| 5 | 430000 | 10000 |
| 6 | 370000 | 0 |
| 7 | 460000 | 10000 |
| 8 | 350000 | 10000 |
| 9 | 300000 | 10000 |
| 10 | 270000 | 0 |
| 11 | 260000 | 10000 |
| 12 | 250000 | 10000 |
| 13 | 230000 | 10000 |
| 14 | 170000 | 0 |
| 15 | 160000 | 10000 |
| 16 | 250000 | 10000 |
| 17 | 460000 | 10000 |
| 18 | 470000 | 0 |
| 19 | 340000 | 10000 |
| 20 | 250000 | 0 |
| 21 | 210000 | 0 |
| 22 | 120000 | 10000 |
| 23 | 990000 | 10000 |
| 24 | 190000 | 10000 |
| 25 | 80000 | 0 |
| 26 | 720000 | 0 |
| 27 | 600000 | 0 |
| 28 | 550000 | 10000 |
| 29 | 350000 | 10000 |
| 30 | 390000 | 10000 |
| 31 | 850000 | 0 |
| 32 | 550000 | 10000 |
| 33 | 460000 | 0 |
| 34 | 380000 | 10000 |
| 35 | 110000 | 0 |
| 36 | 560000 | 0 |

Sample No. 36 indicates a control, i.e., the initial cell concentration of a liquid drug.

Typical results of the MTT method are shown in FIGS. 15A to 16D, indicating the cell proliferation velocity over time after injection.

According to the test results of a trypan blue staining method, the cell concentration of a liquid drug (sample No. 11) before an injection operation (living cell concentration: 560,000 (cells/ml); the number of dead cells: 0 (cells/ml)) fell into a living cell concentration range of 80,000 to 990,000 (cells/ml) for each sample immediately after injection. Therefore, it was demonstrated that instantaneous cell death does not occur immediately after Injection at an injection velocity.

In order to observe damage of cells over time due to an injection operation, the MTT method was used to assess the cell proliferation ability. In this case, some of the flow rates described in Table 2 were used as follows.

| | | | |
|---|---|---|---|
| 1) | 5 ml/min (system) | I | 3-ml syringe |
| 2) | 5 ml/min (system) | I | 5-ml syringe |
| 3) | 9 ml/min (system) | I | 3-ml syringe |
| 4) | 9 ml/min (system) | I | 5-ml syringe |
| 5) | 20 ml/min (system) | I | 3-ml syringe |
| 6) | about 38 ml/min (manual injection) | I | 1-ml syringe |
| 7) | 5 ml/min (system) | II | 3-ml syringe |
| 8) | 5 ml/min (system) | II | 5-ml syringe |
| 9) | 9 ml/min (system) | II | 3-ml syringe |
| 10) | 9 ml/min (system) | II | 5-ml syringe |
| 11) | about 38 ml/min (manual injection) | II | 1-ml syringe |
| 12) | control (no injection operation) | | |

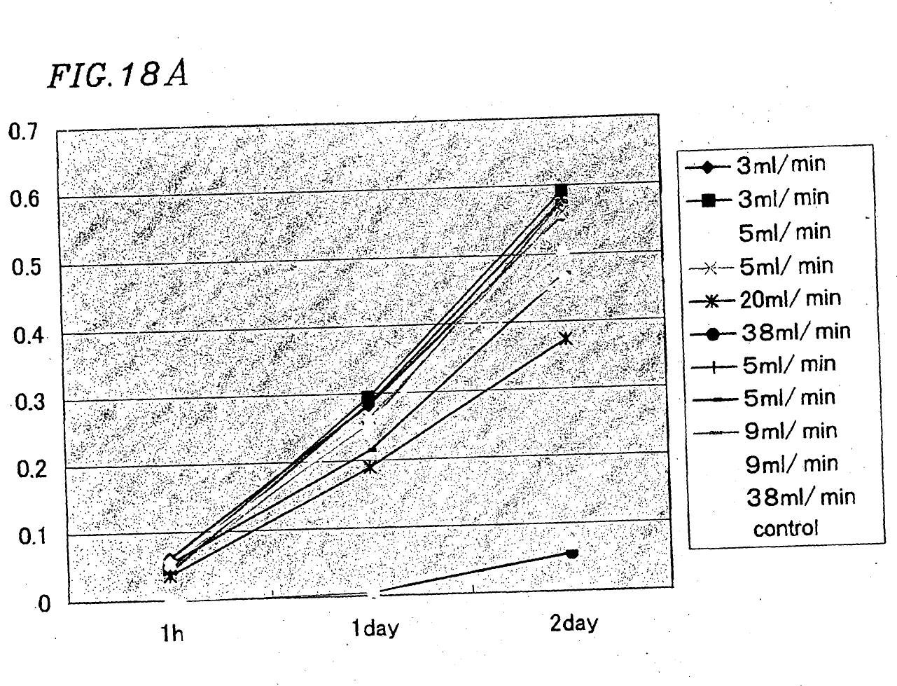

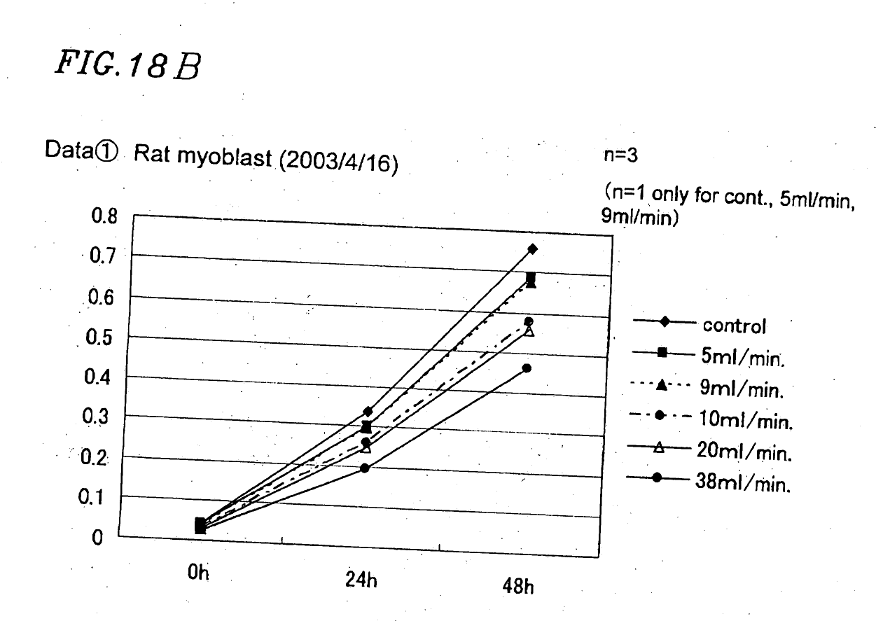

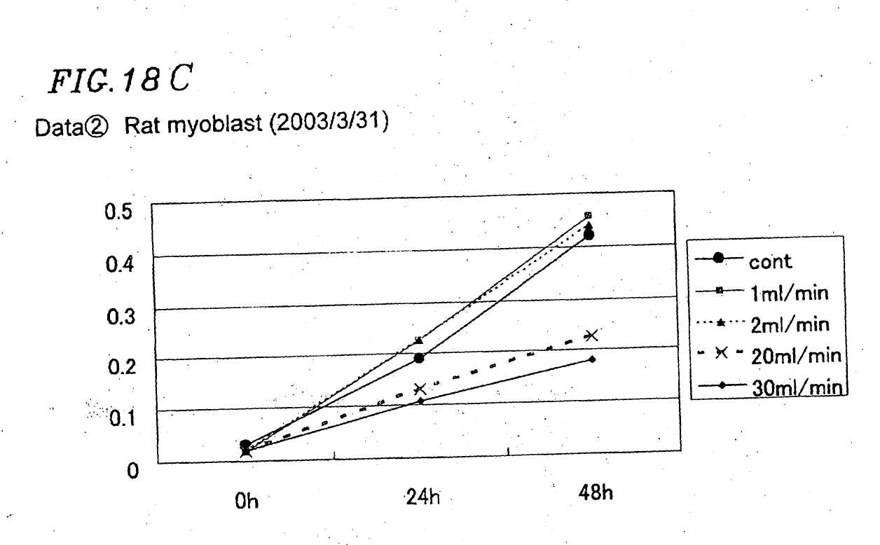

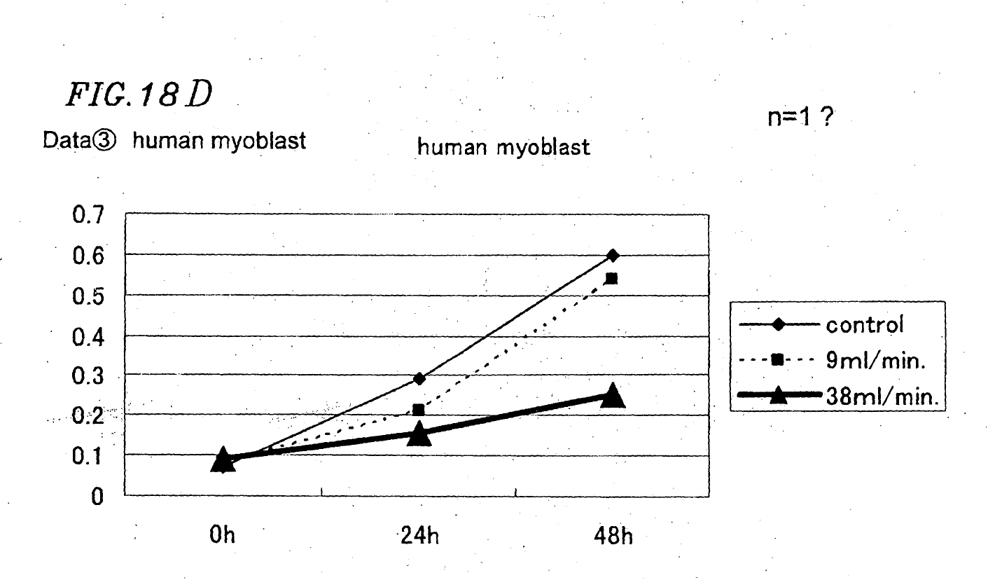

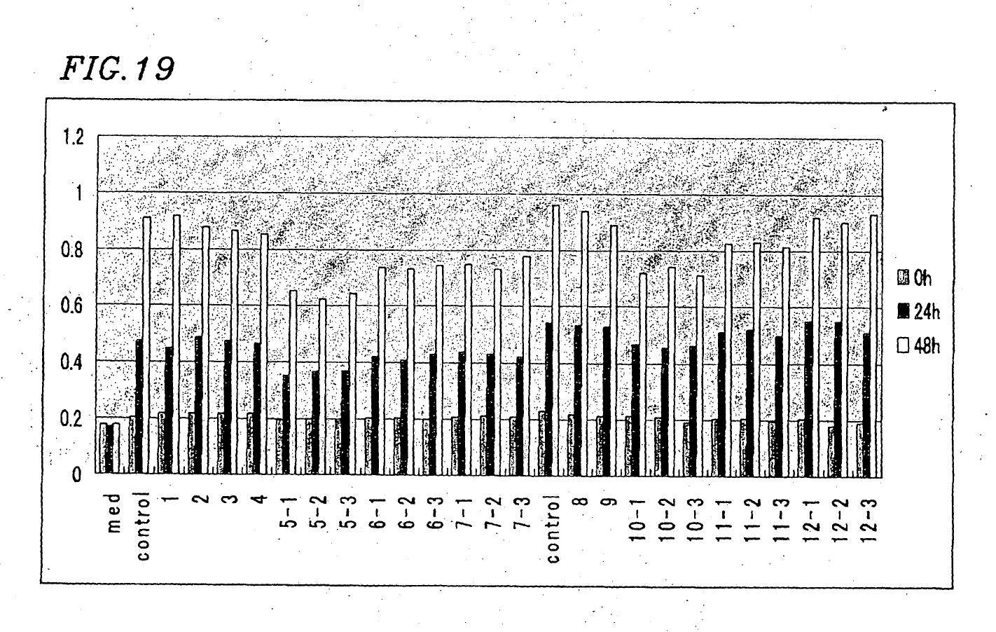

The results are shown in FIG. 18A. After the injection operation, there were a group having substantially the same cell proliferation ability and a group having less proliferation ability. The former was a condition for injection using a system of the present invention. The latter was a condition for injection using a 1-ml syringe (injection velocity: 38 ml/min).

Another system was used to carry out a similar experiment under the following conditions.

| | | | |
|---|---|---|---|
| 1) | control (no injection operation) | I | 5-ml syringe n = 1 |
| | (system) | | |
| 2) | 5 ml/min (system) | I | 5-ml syringe n = 1 |
| 3) | 9 ml/min (system) | I | 5-ml syringe n = 1 |
| 4) | 10 ml/min (system) | I | 5-ml syringe n = 3 |
| 5) | 20 ml/min (system) | I | 5-ml syringe n = 3 |
| 6) | 38 ml/min (manual injection) | I | 1-ml syringe n = 3 |

The results are shown in FIG. 18B. It was found that when the flow rate was less than 10 ml/min, substantially the same cell proliferation ability as that of the control (no injection operation) was maintained. Even when the flow rate was less than or equal to 20 ml/min, the cell proliferation ability was significantly increased as compared to conventional manual injection.

Another system was used to carry out a similar experiment under the following conditions.

| | | | |
|---|---|---|---|
| 1) | control (no injection operation) | I | 5-ml syringe n = 3 |
| | (system) | | |
| 2) | 1 ml/min (system) | I | 5-ml syringe n = 3 |
| 3) | 2 ml/min (system) | I | 5-ml syringe n = 3 |
| 4) | 20 ml/min (system) | I | 5-ml syringe n = 3 |
| 5) | 38 ml/min (manual injection) | I | 1-ml syringe n = 3 |

The results are shown in FIG. 18C. It was found that if the flow rate is less than 10 ml/min, no problem arises. Therefore, only an upper velocity limit should be noted in the present invention. As to the lower limit of the flow rate, only an excessively-long injection time should be avoided.

In addition, human myoblasts were cultured and employed to assess the influence of injection on cell proliferation under the following flow rate conditions.

| | | | |
|---|---|---|---|
| 1) | control (no injection) | I | 5-ml syringe n = 1 |
| 2) | 9 ml/min (system) | I | 5-ml syringe n = 1 |
| 3) | 38 ml/min (manual injection) | I | 1-ml syringe n = 1 |

The results are shown in FIG. 18D. Also for human cells, it was found that if injection is carried out at a flow rate of less than 10 ml/min, the cell proliferation ability is not substantially lowered.

According to the above-described results, it can be said that the proliferation ability of a cell is not lowered, i.e., the cell is not damaged, when the cell is injected with the system of the present invention. However, it was found that when injection was carried out at a flow rate exceeding a predetermined velocity (1-ml syringe, about 38 ml/min), the proliferation ability was reduced to about 20% of the typical level. In contrast, when the flow rate was less than or equal to 20 ml/min, cell injection could be carried out while maintaining the cell proliferation ability without impairing the therapeutic effect. To be summarized, it was found that cells are not substantially damaged if the velocity range thereof is about 20 ml or less. In addition, it was found that the proliferation rate of a cell is not affected if the velocity is less than about 10 ml/min.

It was unexpectedly found that the effect of the predetermined range of velocity does not depend on the type of a syringe, and substantially not on the inner diameter of a syringe. Therefore, the present invention provides an unexpected technique in which by maintaining the velocity (i.e., flow rate) of a cell within a predetermined range, the cell can be injected while maintaining the proliferation ability thereof. This phenomenon has not been heretofore reported and is substantially unpredictable from a physical theory. Thus, the effect of the present invention is significant.

Velocity is the only parameter which has an influence on the result. The velocity can be easily adjusted by physicians or medical practitioners with ordinary skill. Therefore, the present invention provides a technique for simply and efficiently injecting cells into the body.

(Example 2)

Various calls were subjected to injection experiments. In clinical applications, myoblasts, bone marrow cells, and fibroblasts are considered to have high possibility to be actually delivered to the heart among human cells. In this example, rat fibroblasts were used to carry out experiments for modeling the above-described human cells. Fibroblasts are known to be relatively resistant to shock. Therefore, in the present invention, fibroblasts were used as a control in order to investigate the lowest level. These cells were injected using a device as in Example 1. Thereafter, cell death and damage to cell proliferation were studied. The survival states of the cells were determined by trypan blue staining.

Cell-containing liquid drug samples employed are described below.

Rat fibroblasts (primary culture cells) were maintained in a medium (DMEM (High Glucose) (Gibco), supplemented with 2 mM L-glutamine, 50 units/mL penicillin and 50 μg/mL streptomycin) in 5% CO₂ at 37° C.

Test Conditions

TABLE 4

| No. | Injection velocity | Device | Syringe size |
|---|---|---|---|
| 1 | 15 ml/min (system) | I | 20-ml syringe |
| 2 | 20 ml/min (system) | I | 20-ml syringe |
| 3 | 25 ml/min (system) | I | 20-ml syringe |
| 4 | 30 ml/min (system) | I | 20-ml syringe |
| 5 | 35 ml/min (manual injection | I | 20-ml syringe |
| | with syringe) | | |
| 6 | FLASH (system) | I | 20-ml syringe |
| 7 | control no injection operation | | |

Similarly, the proliferation velocity of the cells was measured. In the case of 20 ml/min, substantially no damage was found on the cell. In contrast, in the case of the flow rate of more than 20 ml/min, slight damage was found. In the case of 35 ml/min, the proliferation rate was reduced to about 20% of the typical level as in Example 1.

According to the above-described results, it was demonstrated that when a liquid drug containing a biological material, such as a cell or the like, is injected into organisms, the acceleration and/or velocity of the cell have to be maintained within a predetermined range in order to suppress damage. In particular, a velocity of 20 ml/min or less is effective for any cell.

(Example 3: Further Study on Cell Injection Velocity-Proliferation Curve)

An influence of cell injection velocity on a cell proliferation curve was studied in accordance with the protocol described in Examples 1 and 2 under conditions described in Table 5.

TABLE 5

| | Cell | | Injection | |
|---|---|---|---|---|
| Cell | Density | Syringe | velocity | Condition |
| Rat | 5 × 10⁵ cells/ml | — | — | control |
| myoblast | | 5 cc | 5 ml/min | 1 |
| | | | 9 ml/min | 2 |
| | | 3 cc | 5 ml/min | 3 |
| | | | 9 ml/min | 4 |
| | | 1 cc | 38 ml/min | 5-1 |
| | | | | 5-2 |
| | | | | 5-3 |
| | | | 20 ml/min | 6-1 |
| | | | | 6-2 |
| | | | | 6-3 |
| | | | 10 ml/min | 7-1 |
| | | | | 7-2 |
| | | | | 7-3 |
| | 1 × 10⁷ cells/ml | — | — | control |
| | | 3 cc | 5 ml/min | 8 |
| | | | 9 ml/min | 9 |
| | | 1 cc | 38 ml/min | 10-1 |
| | | | | 10-2 |
| | | | | 10-3 |
| | | | 20 ml/min | 11-1 |
| | | | | 11-2 |
| | | | | 11-3 |
| | | | 10 ml/min | 12-1 |
| | | | | 12-2 |
| | | | | 12-3 |

The results are shown in FIG. 19. As can be seen from the results, when the flow rate is 38 ml/min, proliferation is significantly poor. In contrast, when the flow rate is 20 ml/min or less, the proliferation curve is significantly improved.

(Influence of Tip Tube)

Next, an influence of the diameter of a shaft (tip tube) was measured. Shafts (tip tubes) having an inner diameter in the range of 0.1 mm to 1 mm were employed. In each case, velocity as described in Examples 1 and 2 could be achieved. It was found that the inner diameter of the shaft (tip tubes) has substantially no influence on the proliferation rate of a cell. Therefore, it was unexpectedly demonstrated that in the present invention, cells can be injected into organisms substantially irrespective of the inner diameter of a shaft (tip tubes).

(Example 4)

To understand the relationship between the set value of each injection flow rate and an actual output flow rate, the following experiment was carried out.

Measurement method: the output flow rate of water was measured when a system of the present invention was used to perform an injection operation at a constant flow rate.

Experimental Samples:
(i) Inner diameter: φ0.38 mm; full-length: 1500 mm: PTFE tube (with 27G needle)
(ii) Inner diameter: φ0.30 mm: full-length: 1500 mm: PTFE tube (with 27G needle)
(iii) Inner diameter: φ0.46 mm: full-length: 1500 mm; PTFE tube (with 27G needle)
(iv) Inner diameter: φ0.38 mm; full-length: 1500 mm: PTFE tube (without needle)

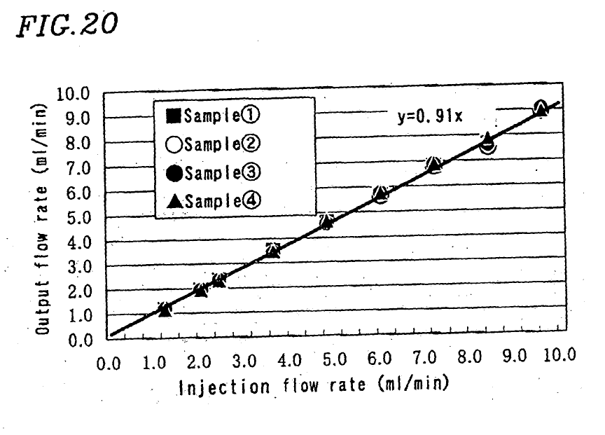

The results of the experiment-are shown in Table 6 below and FIG. 20.

TABLE 6

| | | Injection flow rate (ml/min) | | | | | | | | |
|---|---|---|---|---|---|---|---|---|---|---|
| | | 1.2 | 2.0 | 2.4 | 3.6 | 4.8 | 6.0 | 7.2 | 8.4 | 9.4 |
| Output flow rate | i | 1.163 | 1.938 | 2.330 | 3.503 | 4.609 | 5.671 | 6.810 | 7.666 | 8.955 |
| (ml/min) | ii | 1.173 | 1.929 | 2.307 | 3.407 | 4.557 | 5.636 | 6.795 | 7.528 | 8.937 |
| | iii | 1.187 | 2.008 | 2.381 | 3.498 | 4.555 | 5.753 | 6.860 | 7.624 | 9.050 |
| | iv | 1.161 | 1.938 | 2.315 | 3.467 | 4.679 | 5.775 | 6.897 | 7.895 | 8.951 |

According to the above-described results, the relational expression between the set value of the injection velocity of the system and the actual output flow rate, was obtained. It was clarified that the actual output flow rate was slightly delayed with respect to the set value of the injection flow rate of the system. It is clarified that substantially no influence of such a delay has to be taken into consideration. In addition, according to the result, it was found that a reduction in the flow rate did not vary depending on the inner diameter of the samples used in the experiment or the presence or absence of a needle.

(Example 5)

Next, animal models were used to carry out injection experiments. In conventional cardiac surgery, injection is performed by mapping from the inside of the ventricle to the inside of the heart. A device for three-dimensional mapping costs several tens of millions yen, and requires a high level of skill. Therefore, it is difficult to handle such a device (they say that only 30 people can manipulate the device in Japan). The surgery requires at least two hours. A needle cannot be fixed during injection. The needle receives a great reaction force from a cardiac muscle, so that it is difficult to insert the device into the cardiac muscle. There is the risk of hemorrhage or wall penetration. Therefore, the surgery cannot be said to be stable. Also, arrhythmia has been reported.

The present invention was applied to a coronary artery. Coronary artery can be used if a percutaneous transluminal coronary angioplasty (PTCA) technique is available. Therefore, the scope of applications of the present invention is considered to be wide for coronary arteries. Coronary artery can be used as a rail in the three-dimensional space to determine a precise position using a balloon catheter.

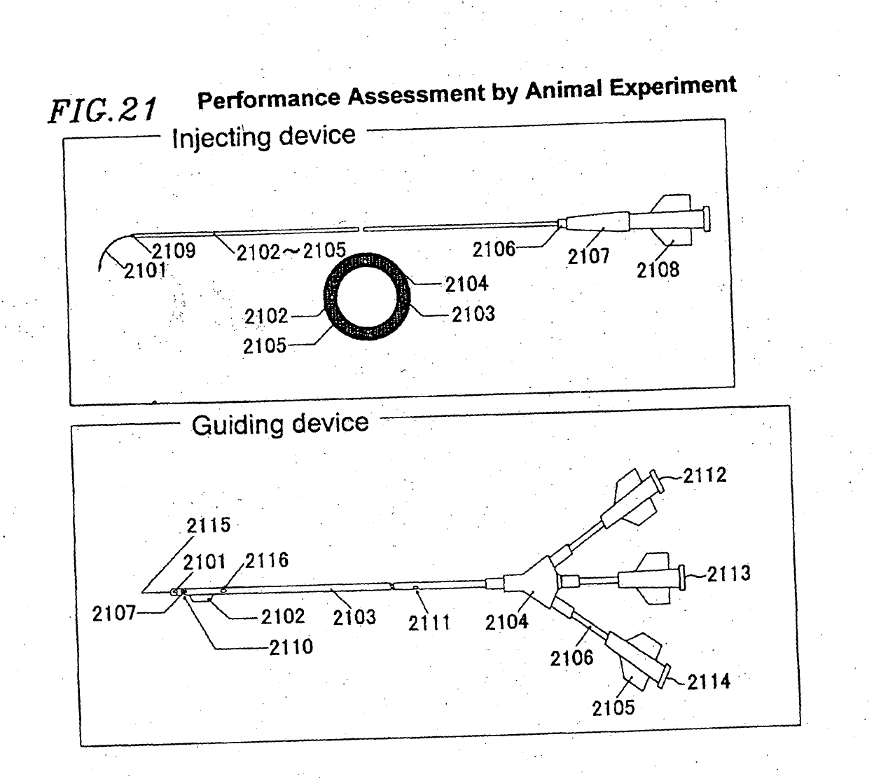

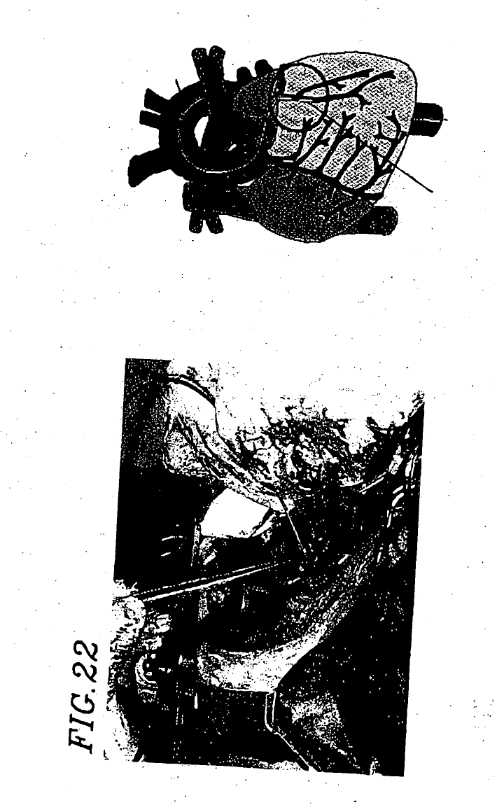

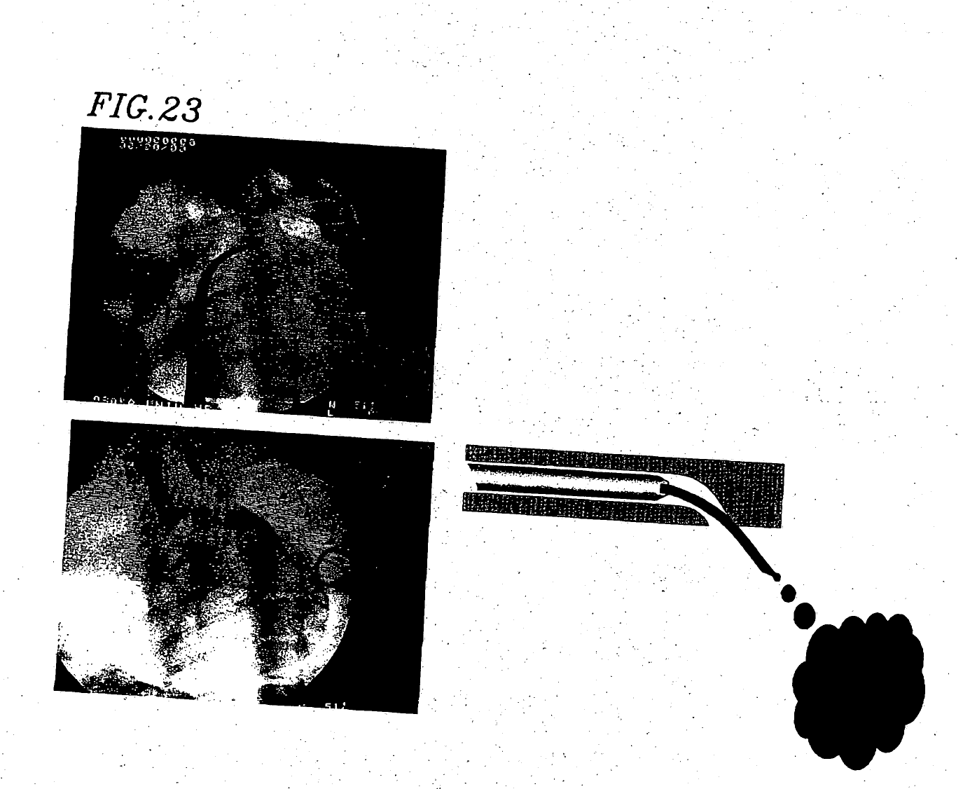

Hereinafter, a procedure in this example will be described in detail. A device shown in FIG. 21 is employed. FIGS. 22 and 23 show how the device was inserted.

A beagle dog weighing 6 kg was systemically anesthetized and a midline incision, was made in the chest. An approach was made through the brachiocephalic trunk. A wire could be inserted into the left coronary artery in a predetermined perspective way. An injection catheter was inserted into the left coronary artery inlet. Since the injection catheter was thicker than the dog coronary artery, the catheter was inserted to a length of only 1.5 cm. A balloon was inserted toward a heart chamber, and an injection needle was projected. A pool of contrast medium was observed at a site in which the needle was projected. The coronary artery was imaged via this leaked contrast medium. To, simulate clinical applications, a pig weighing 40 kg was used to carry out an experiment. The 40-kg pig was systemically anesthetized and a catheter was inserted through a puncture in the femoral artery. After a coronary artery was identified, the catheter was inserted into the coronary artery. A balloon was inserted toward a heart chamber. An injection needle was projected. A pool of contrast medium was observed at a site in which the needle was projected. It was confirmed that the coronary artery was imaged due to the leaked contrast medium.

(Results)

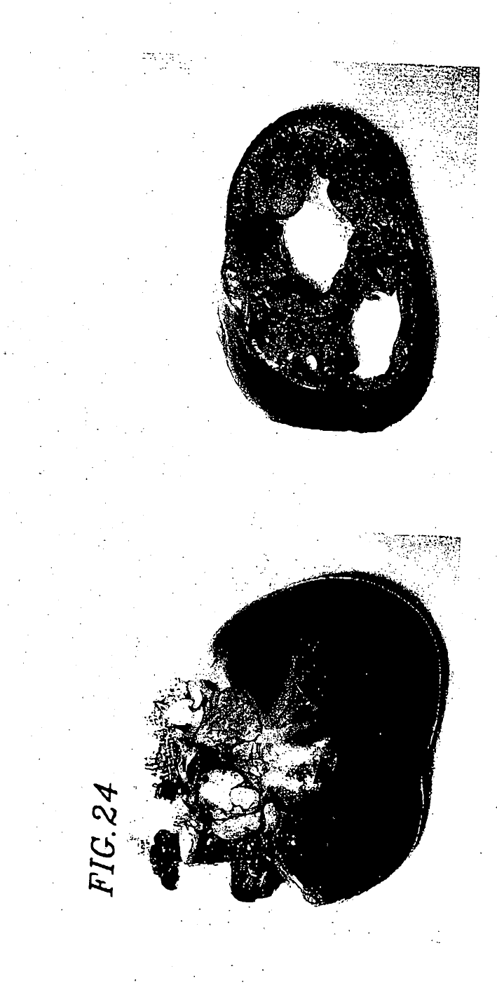

The results of the above-described example are shown in FIG. 24. The beagle dogs were sacrificed. The heart was observed through the outer surface thereof. As can be seen, from FIG. 24, it was confirmed that the needle did not break through the outer surface to leak a staining liquid, because the needle was directed otherwise. It was also confirmed that a green staining liquid was present in a cardiac muscle tissue which seemed to be a site in which the needle was projected.

Thus, in the actual animal experiment, it is demonstrated that the injecting system of the present invention functions efficiently.

(Example 6: Injection Therapy using Liquid Drug Injecting Device)

Next, a liquid drug injecting device of the present invention was used for treatment using cells. A device as shown in FIG. 2 was fabricated. The device was used to carry out a call injection experiment while adjusting velocity in an experimental system as shown in Example 5.

It was confirmed that the device could adjust the cell injection v locity between 1 ml/min and 20 ml/min. The device was used to carry out an experiment as shown in Example 5. Substantially the same result was obtained. It was confirmed that cells could be actually injected efficiently without impairing the survivability of the cells.

Thus, it was confirmed that the device of the present invention could be used to carry out the same experiment as that of Example 5.

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

It will be appreciated that the present invention achieves efficient administration of a composition into a host, which is indispensable for implantation, gene therapy, regeneration medicine, and the like, and the present invention is useful for industries relevant to these fields.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

1. A method for injecting a liquid drug containing a biological material, comprising the step of:
A) injecting the liquid drug containing the biological material contained in an injector into a subject at a predetermined range of velocity.

2. A method according to claim 1, wherein the predetermined range of velocity maintains a biological activity of the biological material.

3. A method according to claim 1, wherein the predetermined range of velocity la less than or equal to about 20 ml/min.

4. A method according to claim 1, wherein the predetermined range of velocity is less than about 10 ml/min.

5. A method according to claim 1, wherein the predetermined range of velocity is greater than or equal to about 1 ml/min and less than about 10 ml/min.

6. A method according to claim 1, further comprising the step of:
B) accelerating the liquid drug containing the biological material at a predetermined range of acceleration to reach the predetermined range of velocity.

7. A method according to claim 6, wherein the predetermined range of acceleration maintains a biological activity of the biological material.

8. A method according to claim 6, wherein the predetermined range of acceleration is in the range of about 1 mm/sec² to about 15 mm/sec².

9. A method according to claim 1, wherein an inner diameter of a body of the Injector is about 1 mm to about 30 mm.

10. A method according to claim 1, wherein an inner diameter of a tip tube of the injector is about 0.1 mm to about 10 mm.

11. A method according to claim 1, wherein the biological material comprises a material selected from the group consisting of nucleic acid molecules, polypeptides, lipids, sugar chains, small organic molecules and complexes thereof, cells, tissues, and organs.

12. A method according to claim 1, wherein the biological material is a cell, and the velocity is about 1 ml/min to about 20 ml/min.

13. A method according to claim 1, wherein the biological material is a cell, and the velocity is about 1 ml/min to about 10 ml/min.

14. A method according to claim 1, further comprising the step of:
C) decreasing a velocity of the liquid drug containing the biological material at a predetermined range of acceleration to substantially zero.

15. A method according to claim 14, wherein the absolute value of an acceleration of the decreasing velocity is in the range of about 1 mm to about 15 mm/sec².

16. A method according to claim 1, wherein the injection is carried out for treatment or prophylaxis of a heart.

17. A method for treating an organ using a liquid drug containing a biological material, comprising the step of:
A) Injecting the liquid drug containing the biological material contained in an injector into a subject at a predetermined range of velocity.

18. A system for injecting a liquid drug containing a biological material, comprising:
A) an injector for injecting the liquid drug containing the biological material to a target organism; and
B) an adjustor for adjusting the injection of the liquid drug containing the biological material so that the injection velocity of the liquid drug containing the biological material can be maintained within a predetermined range.

19. A system according to claim 18, wherein the predetermined range of velocity maintains a biological activity of the biological material.

20. A system according to claim 18, wherein the predetermined range of velocity is less than or equal to about 20 ml/min.

21. A system according to claim 18, wherein the predetermined range of velocity is less than about 10 ml/min.

22. A system according to claim 18, wherein the predetermined range of velocity is greater than or equal to about 1 ml/min and less than about 10 ml/min.

23. A system according to claim 18, wherein the adjustor can accelerate the liquid drug containing the biological material at a predetermined range of acceleration.

24. A system according to claim 23, wherein the predetermined range of acceleration maintains a biological activity of the biological material.

25. A system according to claim 23, wherein the predetermined range of acceleration is in the range of about 1 mm/sec² to about 15 mm/sec².

26. A method according to claim 18, wherein an inner diameter of a body of the injector is about 1 mm to about 30 mm.

27. A method according to claim 18, wherein an inner diameter of a tip tube of the injector is about 0.1 mm to about 10 mm.

28. A system according to claim 18, wherein the adjustor does not have an adverse influence on a material selected from the group consisting of nucleic acid molecules, polypeptides, lipids, sugar chains, small organic molecules and complexes thereof, cells, tissues, and organs.

29. A system according to claim 18, wherein the biological material is a cell, and the velocity is about 1 ml/min to about 20 ml/min.

30. A system according to claim 18, wherein the biological material is a cell, and the velocity is about 1 ml/min to about 10 ml/min.

31. A system according to claim 18, wherein across-sectional area of the injector is about 5 mm² to about 150 mm².

32. A system according to claim 18, wherein, the injection is carried out for treatment or prophylaxis of a heart.

33. A system for treating an organ using a liquid drug containing a biological material, comprising:
A) an injector for injecting the liquid drug containing the biological-material to a target organism; and
B) an adjustor for adjusting the injection of the liquid drug containing the biological material so that the injection velocity of the liquid drug containing the biological material can be maintained within a predetermined range.

34. A liquid drug injecting device, comprising:
a cylinder comprising a nozzle portion at a tip portion thereof, wherein a liquid drug can be loaded into the cylinder and the liquid drug is output through the nozzle portion; and
a pushing portion for pushing out the liquid drug contained in the cylinder through the nozzle portion by external control while maintaining a predetermined velocity substantially unchanged.

35. A liquid drug injecting device according to claim 34, wherein the pushing portion comprises:
a plunger provided with a screw-thread portion arranged around an outer perimeter thereof so that the plunger can be moved into the cylinder; and
a nut-thread portion provided on an inner wall of the cylinder so that the screw-thread portion of the plunger is engaged with the nut-thread portion.

36. A liquid drug injecting device according to claim 34, wherein the pushing portion comprises:
a plunger arranged so that the plunger can be moved into the cylinder; and
a plug provided at a tip portion of the plunger,
wherein the plunger comprises a spring-like elastic member which can be compressed when a velocity or acceleration thereof is greater than or equal to a predetermined value.

37. A liquid drug injecting device according to claim 34, wherein the pushing portion comprises:
a plunger provided in the cylinder; and
an elastic member provided at a tip portion of the plunger,
wherein the elastic member can be compressed when a velocity or acceleration thereof is greater than or equal to a predetermined valued.

38. A liquid drug injecting device according to claim 34, wherein the pushing portion comprises:
a plunger provided with a screw-thread portion on an outer perimeter thereof so that the plunger can be moved into the cylinder;
a nut-thread portion provided on an inner wall of the cylinder so that the screw-thread portion of the plunger is engaged with the nut-thread portion; and
an elastic member provided at a tip portion of the plunger,
wherein the liquid drug contained in the cylinder is pushed out with the tip portion of the plunger by rotating the plunger, and
when the velocity or acceleration of the plunger is greater than or equal to a predetermined value, the elastic member can be compressed.

39. A liquid drug injecting device according to claim 34, wherein the pushing portion comprises:
an inflating member provided on an inner perimeter portion of the cylinder; and
a loading portion for loading an incompressible fluid into the inflating member,
wherein the incompressible fluid is loaded by the loading portion into the inflating member at a substantially constant velocity and/or acceleration thereof.

40. A liquid drug injecting device according to claim. 34, wherein the pushing portion comprises:
a hollow inflating member attached to a rear end portion of the cylinder,
wherein the incompressible fluid is loaded by the loading portion into the inflating member at a substantially constant velocity and/or acceleration thereof.

41. A liquid drug injecting device according to claim 34, wherein the pushing portion comprises:
a plunger movably attached to the cylinder; and
a driving portion for inserting the plunger into the cylinder at a constant velocity.

42. A liquid drug Injecting device according to claim 34, wherein the liquid drug is a liquid containing a cell.
